(19) United States
(12) Patent Application Publication
(10) Pub. No.: US 2022/0032256 A1
(43) Pub. Date: Feb. 3, 2022

(54) DEVICES AND METHODS FOR LIGHT-DIRECTED POLYMER SYNTHESIS

(71) Applicant: Twist Bioscience Corporation, South San Francisco, CA (US)

(72) Inventors: Jeremy LACKEY, Foster City, CA (US); Scott INDERMUEHLE, Danville, CA (US); David DODD, San Francisco, CA (US); Stefan PITSCH, Stein Am Rhein (CH)

(21) Appl. No.: 17/368,697

(22) Filed: Jul. 6, 2021

Related U.S. Application Data
(60) Provisional application No. 63/048,888, filed on Jul. 7, 2020.

(51) Int. Cl. B01J 19/00

(52) U.S. Cl. CPC B01J 19/0046; B01J 2219/00722; B01J 2219/00711; B01J 2219/00596

(57) ABSTRACT

Provided herein are compositions, devices, systems and methods for generation and use of biomolecule-based information for storage. Further provided are devices comprising addressable LED arrays to control polynucleotide synthesis (deprotection, extension, or cleavage, etc.) The compositions, devices, systems and methods described herein provide improved storage, density, and retrieval of biomolecule-based information.

### CROSS-REFERENCE

This application claims the benefit of U.S. Provisional Application No. 63/048,888, filed Jul. 7, 2020, which is incorporated by reference in its entirety.

### BACKGROUND

Light directed synthesis of DNA typically has a light source projected onto the chip using a physical mask or a digital mirror device (DMD). Such processes have a shortcoming of limiting the density of the array by the diffraction limit. There is a need for more scalable, automated, highly accurate and highly efficient systems for generating biomolecules de novo.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

Provided herein are devices for polymer synthesis comprising: a solid support, wherein the solid support comprises a plurality of wells, wherein each of the wells comprises: a) a synthesis surface located in a bottom region of each of the wells; b) a light emitting layer in addressable communication with the synthesis surface and situated below the synthesis surface; and c) a CMOS driver located in addressable communication with the light emitting layer. Further provided herein are devices wherein the light-emitting layer is a light-emitting diode (LED). Further provided herein are devices wherein the LED is an organic LED (OLED). Further provided herein are devices wherein the LED is a micro-LED. Further provided herein are devices wherein the light-emitting layer emits ultraviolet (UV) light. Further provided herein are devices wherein the UV light has a wavelength of about 350 nm. Further provided herein are devices wherein the UV light has a wavelength of about 365 nm. Further provided herein are devices wherein the UV light has a wavelength of about 400 nm. Further provided herein are devices wherein the light-emitting layer emits visible light. Further provided herein are devices wherein the visible light has a wavelength of about 405 nm. Further provided herein are devices wherein the visible light has a wavelength of about 450 nm. Further provided herein are devices wherein the light-emitting layer emits infrared (IR) light. Further provided herein are devices wherein the IR light has a wavelength of about 800 nm. Further provided herein are devices wherein the solid support comprises addressable loci at a density of at least 10×10⁶ addressable loci per cm². Further provided herein are devices wherein the solid support comprises addressable loci at a density of 10×10⁶ to 10⁹ addressable loci per cm². Further provided herein are devices wherein the addressable locus comprises a diameter up to about 1000 nm. Further provided herein are devices wherein each of the wells comprises a depth up to about 1000 nm. Further provided herein are devices wherein each of the wells comprises a depth of 100 nm to 1000 nm. Further provided herein are devices wherein each of the wells comprises a longest cross-sectional diameter of 100 nm to 1000 nm. Further provided herein are devices wherein each of the wells comprises a longest cross-sectional diameter of about 2 um. Further provided herein are devices wherein each of the wells comprises a longest cross-sectional diameter of about 5 um. Further provided herein are devices wherein each of the wells is cylindrical.

Provided herein are methods for synthesizing a polymer, comprising: a) providing a solid support comprising a surface; b) depositing at least one nucleoside on the surface, wherein the at least one nucleoside couples to a polynucleotide attached to the surface, wherein the coupling comprises a light-directed deprotection step by a light-emitting layer, and wherein the light-emitting layer is located beneath the surface; and c) repeating step b) to synthesize a plurality of polynucleotides on the surface, wherein polynucleotides having different sequences on the surface are present at a density of at least 100×10⁶ polynucleotides per cm². Further provided herein are methods wherein the light-emitting layer is a light-emitting diode (LED). Further provided herein are methods wherein the LED is an organic LED (OLED). Further provided herein are methods wherein the LED is a micro-LED. Further provided herein are methods wherein the light-emitting layer emits ultraviolet (UV) light. Further provided herein are methods wherein the UV light has a wavelength of about 350 nm. Further provided herein are methods wherein the UV light has a wavelength of about 365 nm. Further provided herein are methods wherein the UV light has a wavelength of about 400 nm. Further provided herein are methods wherein the light-emitting layer emits visible light. Further provided herein are methods wherein the visible light has a wavelength of about 405 nm. Further provided herein are methods wherein the visible light has a wavelength of about 450 nm. Further provided herein are methods wherein the light-emitting layer emits infrared (IR) light. Further provided herein are methods wherein the IR light has a wavelength of about 800 nm. Further provided herein are methods wherein the solid support comprises addressable loci at a density of at least 10×10⁶ addressable loci per cm². Further provided herein are methods wherein the solid support comprises addressable loci at a density of 10×10⁶ to 10⁹ addressable loci per cm². Further provided herein are methods wherein the addressable locus comprises a diameter up to about 1000 nm. Further provided herein are methods wherein the deprotection step deprotects a 5′-hydroxyl group. Further provided herein are methods wherein the deprotection step removes a nitrophenylpropyloxycarbonyl (NPPOC) protecting group. Further provided herein are methods wherein the deprotection step removes a 2,(3,4-methylenediooxy-6-nitrophenyl)propoxycarbonyl (MNPPOC) group.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the phosphoramidite synthesis cycle in maskless array synthesis (MAS).

FIG. 2A illustrates a simplified cross-section diagram of an organic light emitting diode (OLED) light-directed polymer synthesis device with two reaction chambers. FIG. 2B illustrates a simplified cross-section diagram of a micro-light emitting diode (LED) light-directed polymer synthesis device with two reaction chambers.

FIG. 3A illustrates photon emission of the complementary metal-oxide-semiconductor (CMOS) driver on the left, which illuminates the reaction chamber on the left. FIG. 3B illustrates photon emission of the CMOS driver on the right, which illuminates the reaction chamber on the right.

FIG. 4A illustrates the basic OLED cell structure comprising (bottom to top) a substrate, an anode, hole injection layer (HIL), hole transport layer (HTL), light-emitting layer, blocking layer (BL), electron transport layer (ETL), and cathode. FIG. 4B illustrates the OLED example stack of the disclosure comprising (from top to bottom) an OLED stack, CMOS top metal layer, an interconnection layer, and an active CMOS.

FIG. 5 illustrates an example of a computer system.

FIG. 6 is a block diagram illustrating architecture of a computer system.

FIG. 7 is a diagram demonstrating a network configured to incorporate a plurality of computer systems, a plurality of cell phones and personal data assistants, and Network Attached Storage (NAS).

FIG. 8 is a block diagram of a multiprocessor computer system using a shared virtual address memory space.

FIG. 9A and FIG. 9B show the specifications of the GaN microLED chip. Features are labeled as (top to bottom) oxide, micro-LED, and CMOS drive pixel.

FIG. 10A and FIG. 10B show DC I-V plots of wafers with unroughened and roughened surfaces. The x-axis depict voltage (V) from −5 to 8 volts at 1 volt intervals. The y-axis depict current (A) from 1×10⁻¹¹ to 1×10⁻² on a log scale.

FIG. 11 shows peak external quantum efficiency measurements for 10 wafer samples. The x-axis depicts J(A/cm²) from 1 to 1000 on a log scale. The y-axis depicts EQE from 0.07 to 0.29 at 0.02 intervals.

FIG. 12 (left) shows an image of the packaged chip. FIG. 12 (right) shows an image of a fluidics system required for DNA synthesis.

FIGS. 13A and 13B show UV spectra of 5′-photolabile dT amidites cleavable at 405 nm.

FIG. 14A shows the chemical reactions of the control experiment. FIG. 14B shows the chemical reactions of the proof of concept experiment.

FIG. 15 shows an image of the control reaction performed using on-chip 1 μm microLED DNA synthesis.

FIG. 16 shows that the control reaction resulted in flow cell leakage, and the dye was visualized as the background.

FIG. 17 shows an image of the proof of concept reaction performed using on-chip 1 μm microLED DNA synthesis.

FIG. 18 shows that the proof of concept experiment resulted in dye fluorescence after 1 min exposure with a 4V battery.

### DETAILED DESCRIPTION OF THE INVENTION

Provided herein are methods, compositions, devices and systems for synthesizing biopolymers using light-directed deprotection chemistry. Also provided herein are methods to increase biopolymers synthesis throughput through increased sequence density decreased turn-around time using light-directed polymer synthesis.

### Definitions

Unless defined otherwise, all technical and scientific terms used herein have the same meaning as is commonly understood by one of ordinary skill in the art to which these inventions belong.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein, the terms “preselected sequence”, “predefined sequence” or “predetermined sequence” are used interchangeably. The terms mean that the sequence of the polymer is known and chosen before synthesis or assembly of the polymer. In particular, various aspects of the invention are described herein primarily with regard to the preparation of nucleic acids molecules, the sequence of the polynucleotide being known and chosen before the synthesis or assembly of the nucleic acid molecules.

Provided herein are methods and compositions for production of synthetic (i.e. de novo synthesized or chemically synthesized) biopolymers. Biopolymers include, but are not limited to, polynucleotides, oligonucleotides, peptides, peptide conjugates, oligosaccharides, or any polymer or biomolecule that is synthesized in a controlled fashion. Polynucleotide sequences described herein may be, unless stated otherwise, comprise DNA or RNA. Chemically modified DNA or RNA may include, but is not limited to, 2′-F, 2′-MOE, phosphonothioate, unnatural base-modified, boranophosphonate, or morpholino-modified DNA or RNA.

### Solid Support-Based, Light-Directed Biopolymer Synthesis and Storage

Described herein are methods, compositions, devices, and systems solid support-based, light-directed biopolymer synthesis and storage. In some instances, polynucleotides are de novo synthesized using solid support-based, light-directed methods as described herein. In some instances, polynucleotides are stored on a solid support following light-directed synthesis. In some instances, solid support-based, light-directed methods as described herein are used for storage only.

Described herein are devices, systems, and methods for solid support-based, light-directed biopolymer synthesis and storage, wherein one or more biopolymer synthesizer components are integrated into a solid support. Components or functional equivalents of components may comprise temperature control units, addressable electrodes, semiconducting surfaces (e.g., complementary metal-oxide semiconductor), fluid reservoirs, fluidics, synthesis surfaces, power sources, light-emitting diodes (LEDs), organic light-emitting diodes (OLEDs), or other components used to synthesize polymers. Any combination of integrated components is suitable for use with the devices, systems, and methods described herein. In some instances, one or more components is external (non-integrated) to the solid support.

Coupling in some instances is controlled through the nucleoside addition step, a deprotection step, or other step that affects the efficiency of a nucleoside coupling reaction. In some instances, polynucleotides are deprotected using light-labile deprotection chemistry, making polynucleotides available for coupling to nucleosides.

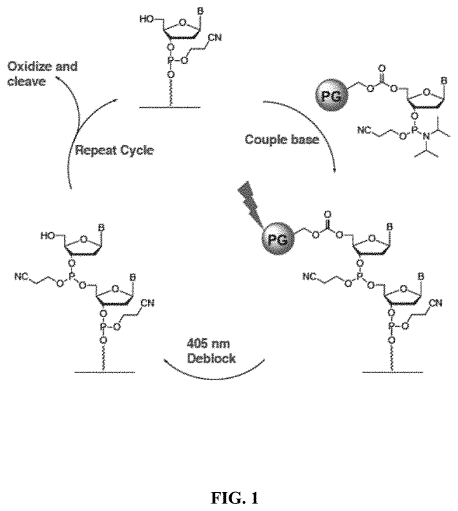

In situ, light-directed biopolymer synthesis uses photolabile 5′-hydroxyl protecting groups in phosphoramidite combinatorial chemistry. FIG. 1 illustrates a phosphoramidite synthesis cycle using photolabile 5′-hydroxyl protecting groups and light-directed deprotection chemistry. The phosphoramidite synthesis cycle is similar to that used in solid-phase synthesis of nucleic acids. UV light (e.g., from the I-line of mercury or a light-emitting diode) in the presence of an organic base is used to deprotect the 5′-OH. Oxidation of the phosphites is not required in the cycle because they are not exposed to acid. The final chemical deprotection step must not cleave the nucleic acids from the surface.

In some instances, microarrays are manufactured using light exposure patterned by physical masks placed over the synthesis surface. In some instances, microarrays use maskless array synthesis (MAS), where a digital micromirror device (DMD) is used in place of photomasks to deliver patterned ultraviolet light. The pattern displayed on the micromirror device is transferred to the synthesis surface, where the array layout and oligonucleotide sequences are determined by selective removal of the photocleavable protecting groups on the 5′-end of the terminal phosphoramidites on the microarray.

Described herein are devices, systems, and methods for biopolymer synthesis comprising a solid support, wherein the solid support comprises a plurality of wells, wherein each of the wells comprises an addressable locus further comprising: a synthesis surface located in a bottom region of each of the wells; a light-emitting layer; and an addressable semiconducting device. The wells of the devices disclosed herein comprise an oxide layer that lays on top of, and in contact with, a light-emitting layer. The light-emitting layer further lays on top, and in contact with, a CMOS driver. The individual CMOS drivers can be controlled to generate a current, which results in photon emission from the light-emitting layer. Photon emission of the light-emitting layer illuminates the reaction chambers to photocleave the 5′-photolabile protecting group on the 5′-end of the terminal phosphoramidites.

The light-directed biopolymer synthesis of the disclosure utilizes 5′-photolabile protecting groups on the 5′-end of terminal phosphoramidites. In some instances, the 5′-photolabile protecting group is nitrophenylpropyloxycarbonyl (NPPOC), 2,(3,4-methylenediooxy-6-nitrophenyl)propoxycarbonyl (MNPPOC), benzoyl-NPPOC, or thiophenyl-NPPOC.

In some instances, the photolabile protecting group can be an ortho-nitrobenzyl derivative, a coumadin derivative, or another chemical protecting group. Exemplary photolabile groups include, but are not limited to:

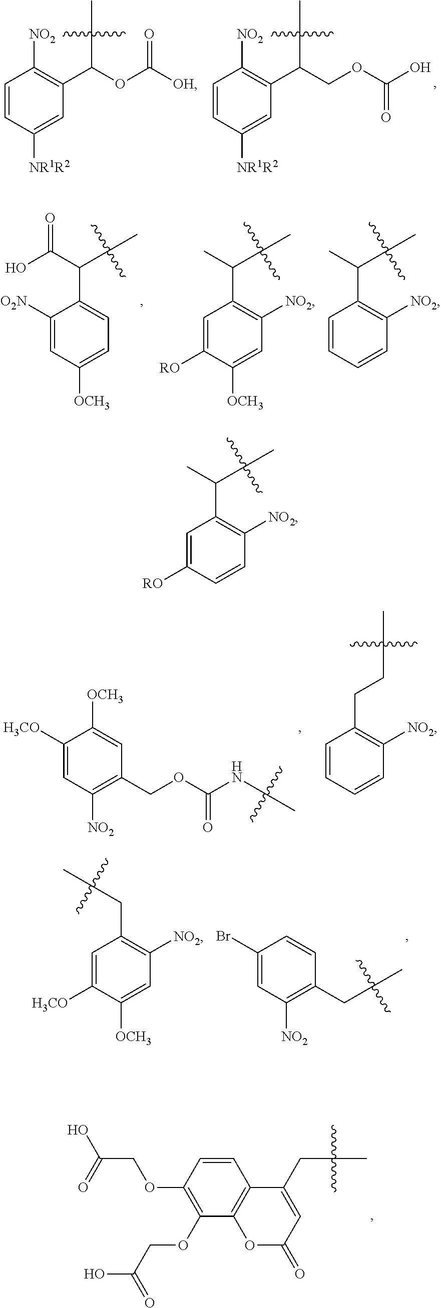

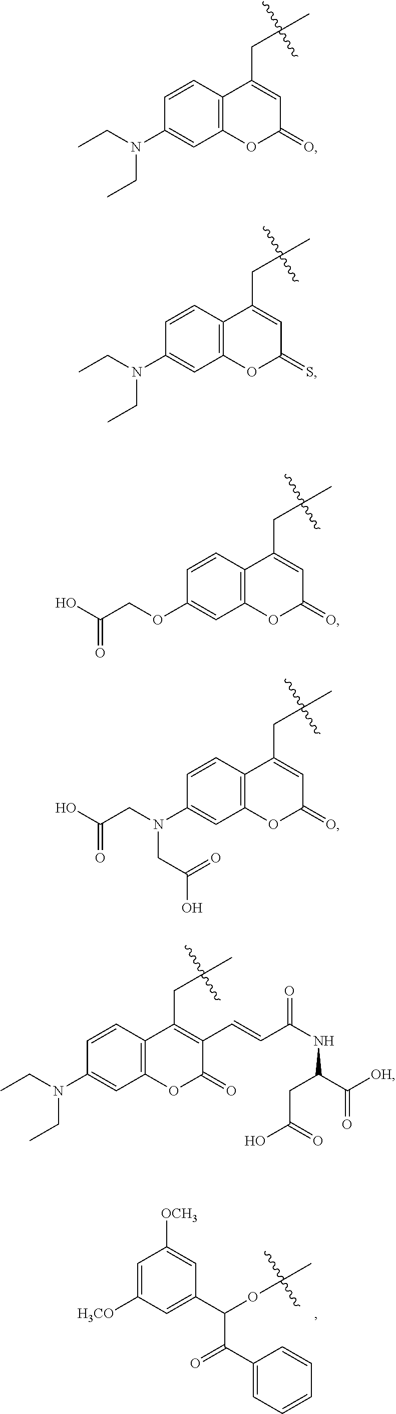

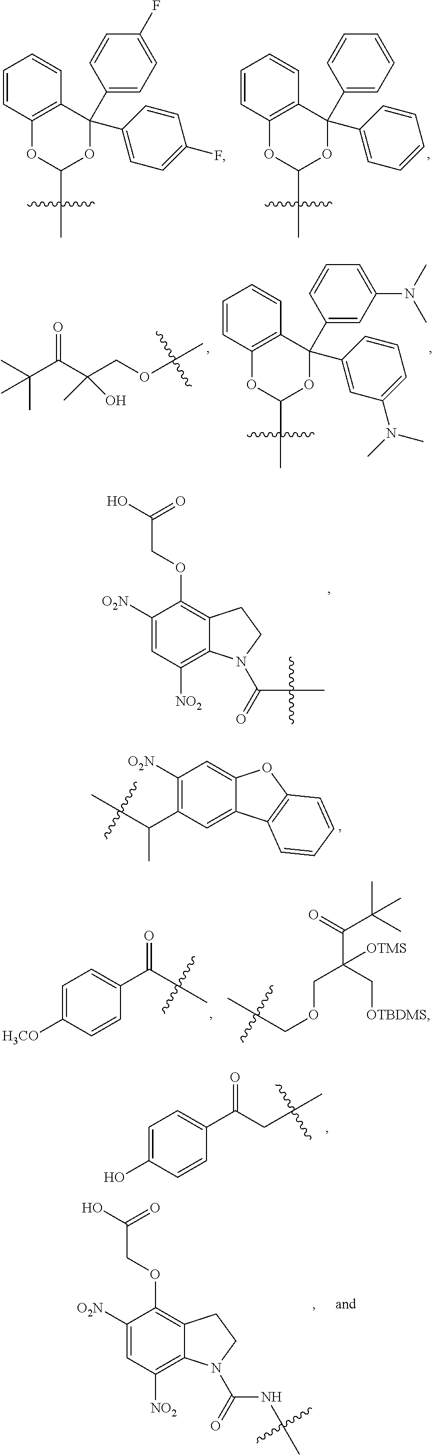

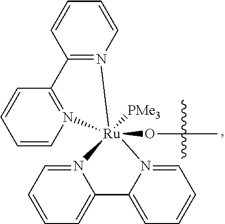

wherein each R, R¹, and R² is independently is selected from a group consisting of: —C(O)R³, —C(O)OR³, —C(O)NR³R⁴, —SOR³, —SO₂R⁴, alkyl, alkenyl, alkynyl, aryl, heteroaryl, or heterocyclyl, each of which is independently substituted or unsubstituted, or hydrogen, or R¹ and R² together with the nitrogen atom to which R¹ and R² are bound form a ring, wherein the ring is substituted or unsubstituted; wherein each R³ and R⁴ is independently —C(O)R⁵, —C(O)OR⁵, —C(O)NR⁵R⁶, —OR⁵, —SR⁵, —NR⁵R⁶, —NR⁵C(O)R⁶, —OC(O)R⁵, alkyl, alkenyl, alkynyl, aryl, heteroaryl, or heterocyclyl, each of which is independently substituted or unsubstituted, or hydrogen or halogen; wherein each R⁵ and R⁶ is independently alkyl, alkenyl, alkynyl, aryl, heteroaryl, or heterocyclyl, each of which is independently substituted or unsubstituted, or hydrogen.

In some embodiments, the photolabile group is

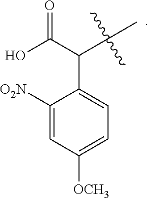 [SMILES: *C(C(=O)O)c1ccc(OC)cc1[N+](=O)[O-]]

In some embodiments, the photolabile group is

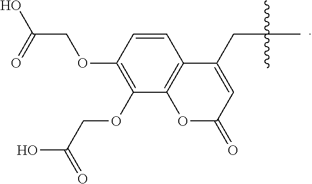 [SMILES: *Cc1cc(=O)oc2c(OCC(=O)O)c(OCC(=O)O)ccc12]

In some embodiments, the photolabile group is

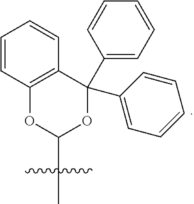 [SMILES: *C1Oc2ccccc2C(c2ccccc2)(c2ccccc2)O1]

In some embodiments, the photolabile group is

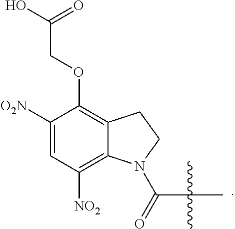 [SMILES: *C(=O)N1CCc2c(OCC(=O)O)c([N+](=O)[O-])cc([N+](=O)[O-])c21]

In some embodiments, the photolabile group is

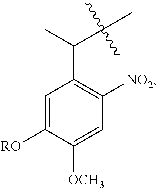

wherein R is —C(O)R³, —C(O)OR³, —C(O)NR³R⁴, —SOR³, —SO₂R⁴, alkyl, aryl, heteroaryl, or heterocyclyl, each of which is independently substituted or unsubstituted, or hydrogen; wherein each R³ and R⁴ is independently alkyl, aryl, heteroaryl, or heterocyclyl, each of which is independently substituted or unsubstituted, or hydrogen. In some embodiments, R is substituted or unsubstituted alkyl. In some embodiments, R is substituted or unsubstituted aryl. In some embodiments, R is substituted or unsubstituted heteroaryl.

Exemplary photolabile groups also include, but are not limited to:

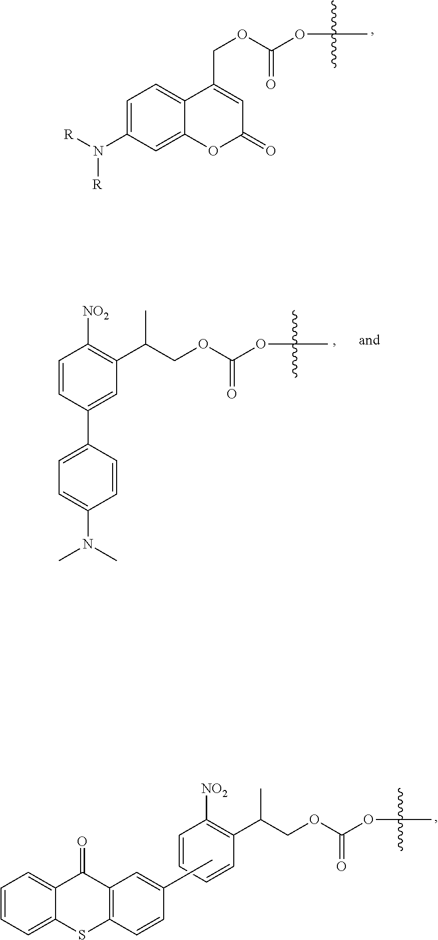

wherein R is a variable as defined above.

Provided here are light-directed biopolymer synthesis methods which include a dose ranging from 0.01 J/cm² to 100 J/cm² to photocleave the photolabile protecting group. In some instances, the photolabile protecting groups are photocleaved using 1 J/cm² to 20 J/cm² of light. In some instances, the photolabile protecting groups are photocleaved using 1 J/cm², 2 J/cm², 3 J/cm², 4 J/cm², 5 J/cm², 6 J/cm², 7 J/cm², 8 J/cm², 9 J/cm², or 10 J/cm² of light. In some instances, the photolabile protecting groups are photocleaved using 4 J/cm², 5 J/cm², 6 J/cm², 7 J/cm², or 8 J/cm² of light. In some instances, the photolabile protecting groups are photocleaved using 5 J/cm² or 6 J/cm² of light.

Provided herein are light-directed biopolymer synthesis methods where cleavage of photolabile protecting groups is performed by applying electromagnetic radiation (EMR) at ultraviolet (UV), visible light, or IR wavelengths. The EMR at UV, visible light, or IR wavelengths are provided by the light emitting layer of the disclosed device, for example, an LED or OLED. The photolabile protecting group is cleaved by applying EMR at a wavelength from about 100 nm to about 800 nm, from about 100 nm to about 400 nm, or from about 200 nm to about 300 nm.

In some instances, the photolabile protecting group is cleaved by applying EMR at UV wavelengths. In some instances, the photolabile protecting group is cleaved by applying EMR at a UV wavelength from about 300 nm to about 400 nm. In some instances, EMR is applied at a wavelength of about 300 nm. In some instances, EMR is applied at a wavelength of about 350 nm. In some instances, EMR is applied at a wavelength of about 365 nm. In some instances, EMR is applied at a wavelength of about 400 nm. In some instances, the photolabile protecting group is cleaved by applying EMR at visible wavelengths. In some instances, the photolabile protecting group is cleaved by applying EMR at a wavelength from about 400 nm to about 800 nm. In some instances, EMR is applied at a wavelength of about 405 nm. In some instances, EMR is applied at a wavelength of about 450 nm. In some instances, EMR is applied at a wavelength of about 500 nm. In some instances, the photolabile protecting group is cleaved by applying EMR at IR wavelengths. In some instances, the photolabile protecting group is cleaved by applying EMR at a wavelength of about 800 nm.

Provided herein are light-directed biopolymer synthesis methods where light emission systems comprise a CMOS driver and light-emitting layer, which are fabricated of materials well known in the art. Materials may comprise metals, non-metals, mixed-metal oxides, nitrides, carbides, silicon-based materials, or other materials.

Light emission systems can possess any shape, including discs, rods, wells, posts, a substantially planar shape, or any other form suited for biopolymer synthesis. The or cross-sectional area of each light emission system varies as a function of the size of the loci for biopolymer synthesis, but in some instances is up to 500 um², 200 um², 100 um², 75 um², 50 um², 25 um², 10 um², or less than 5 um². In some instances, the cross-sectional area of each light emission system is about 500 um² to 10 um², about 100 um² to 25 um², or about 150 um² to 50 um². In some instances, the cross-sectional area of each light emission system is about 150 um² to 50 um².

Devices provide herein include light emission systems having a diameter that varies as a function of the size of the loci for biopolymer synthesis. Exemplary light emission system diameters include, without limitation, up to 500 um, 200 um, 100 um, 75 um, 50 um, 25 um, 10 um, or less than 5 um. In some instances, the diameter of each light emission system is about 500 um to 10 um, about 100 um to 25 um, about 100 um to about 200 um, about 50 um to about 200 um, or about 150 um to 50 um. In some instances, the diameter of each light emission system is about 200 um to 50 um. In some instances, the diameter of each light emission system is about 200 um to 100 um. In some instances, the diameter of each light emission system is up to 500 nm, 200 nm, 100 nm, 75 nm, 50 nm, 25 nm, 10 nm, or less than 5 nm. In some instances, the diameter of each light emission system is about 500 nm to 10 nm, about 100 nm to 25 nm, about 100 nm to about 200 nm, about 50 nm to about 200 nm, or about 150 nm to 50 nm. In some instances, the diameter of each light emission system is about 200 nm to 50 nm. In some instances, the diameter of each light emission system is about 200 nm to 100 nm.

The thickness of each light emission system varies as a function of the size of the loci for biopolymer synthesis, but in some instances is about 10 nm, 20 nm, 30 nm, 40 nm, 50 nm, 100 nm, 200 nm, 500 nm, 750 nm, 1000 nm, 1200 nm, 1500 nm, 2000 nm, 2500 nm, 3000 nm, or about 3500 nm. In some instances, the thickness of the light emission system is at least 50 nm, 100 nm, 200 nm, 500 nm, 750 nm, 1000 nm, 1200 nm, 1500 nm, 2000 nm, 2500 nm, 3000 nm, or at least 3500 nm. In some instances, the thickness of the light emission system is at least 1 um, 2 um, 3 um, 5 um, 10 um, 15 um, 20 um, 30 um, 50 um or at least 75 um. In some instances the thickness of the light emission system is about 1 um, 2 um, 3 um, 5 um, 10 um, 15 um, 20 um, 30 um, 50 um or about 75 um. In some instances the thickness of the light emission system is up to 1 um, 2 um, 3 um, 5 um, 10 um, 15 um, 20 um, 30 um, 50 um or up to 75 um. In some instances, the thickness of the light emission system is up to 50 nm, 100 nm, 200 nm, 500 nm, 750 nm, 1000 nm, 1200 nm, 1500 nm, 2000 nm, 2500 nm, 3000 nm, or up to 3500 nm. In some instances, the thickness of the light emission system is about 20 nm to 3000 nm, about 50 nm to 2500, about 100 nm to 750 nm, about 400 nm to 750 nm, about 500 nm to 3000 nm, or about 1000 nm to 3000 nm. In some instances, the thickness of the light emission system is about 10 um to about 20 um. In some instances, the thickness of the light emission system is about 5 um to about 50 um, about 10 um to about 30 um, about 15 um to about 25 um, or about 30 um to about 50 um. In some instances, light emission systems are coated with additional materials such as semiconductors or insulators. In some instances, light emission systems are coated with materials for biopolymer attachment and synthesis.

Each light-emission system can control one or a plurality of different loci for biopolymer synthesis, wherein each locus for synthesis has a density of biopolymers. In some instances, the biopolymer is a polynucleotide, and the density is at least 1 oligo per 10 nm², 20 nm², 50 nm², 100 nm², 200 nm², 500 nm², 1,000 nm², 2,000 nm², 5,000 nm² or at least 1 oligo per 10,000 nm². In some instances, the biopolymer is a polynucleotide, and the density is about 1 oligo per 10 nm² to about 1 oligo per 5,000 nm², about 1 oligo per 50 nm² to about 1 oligo per 500 nm², or about 1 oligo per 25 nm² to about 1 oligo per 75 nm². In some instances, the biopolymer is a polynucleotide, and the density of polynucleotides is about 1 oligo per 25 nm² to about 1 oligo per 75 nm².

The duration of each step in the synthesis cycle can range from 100 ms-2 min. In some instances, the duration of each step in the synthesis cycle can range from 100 ms-500 ms. In some instances, the duration of each step in the synthesis cycle can range from 500 ms-800 ms. In some instances, the duration of each step in the synthesis cycle can range from 30 secs-60 secs. In some instances, the light deprotection step is about 20 secs, 30 secs, 40 secs, 50 secs, 60 secs, 70 secs, 80 secs, or 90 secs. In some instances, the light deprotection step is about 40 secs, 50 secs, 60 secs, 70 secs, or 80 secs. In some instances, the light deprotection step is about 60 secs.

Movement of fluids in or out of surfaces described herein may comprise modifications or conditions that prevent unwanted fluid movement or another phenomenon. For example, fluid movement in some instances results in the formation of bubbles or pockets of gas, which limits contact of fluids with components such as surfaces or polynucleotides. Various methods to control or minimize bubble formation are contemplated by the methods, and systems described herein. Such methods include control of fluid pressure, well geometry, or surface materials/coatings. Well geometry can be implemented to minimize bubbles. For example, tapering the well, channels, or other surface can reduce or eliminate bubble formation during fluid flow. Surface materials possessing specific wetting properties can be implemented to reduce or eliminate bubble formation. For example, surfaces described herein comprise hydrophobic materials. In some instances, surfaces described herein comprise hydrophilic materials.

Pressure can be used to control bubble formation during fluid movement. Pressure in some instances is applied locally to a component, an area of a surface, a capillary/channel, or applied to an entire system. Pressure is in some instances applied either behind the direction of fluid movement, or in front of it. In some instances, back pressure is applied to prevent the formation of bubbles. Suitable pressures used for preventing bubble formation can range depending on fluid, the scale, flow geometry, and the materials used. For example, 5 to 10 atmospheres of pressure are maintained in the system. In some instances, at least 2, 3, 4, 5, 6, 7, 8, 9, 10, 11, 12, 15, 20 or more than 50 atmospheres of pressure are applied. In some instances, up to 2, 3, 4, 5, 6, 7, 8, 9, 10, 11, 12, 15, 20 or up to 50 atmospheres of pressure are applied. In some instances, about 2 to about 10, about 2 to about 8, about 2 to about 5, about 4 to about 10, about 4 to about 12, about 5 to about 15, about 5 to about 7, about 7 to about 20, about 8 to about 15, or about 10 to about 20 atmospheres of pressure are applied.

Devices described herein may utilize control units for the purpose of regulating environmental conditions, such as temperature. Temperature control units are often used to prepare or maintain conditions for storing solid supports comprising biopolymers. Storage conditions of biopolymers can affect their long-term stability, which directly influences the quality of the digital storage information that is retrieved. Biopolymers are optionally stored at low temperature (for example, 10 degrees C., 4 degrees C., 0 degrees C., or lower) on a solid support, wherein a temperature control unit maintains this solid support temperature. The storage medium for biopolymers on a solid support, such as solvated or dry also influences storage stability. In some instances, the biopolymer is a polynucleotide, and is stored in solution, such as an aqueous solution or buffer in droplets. In some instances, the biopolymer is a polynucleotide, and is stored lyophilized (dry).

Temperature control units in some instances increase the chip temperature to facilitate drying of biopolymers attached thereto. Temperature control units also provide for local control of heating at addressable locations on the solid support in some instances. In some instances, following addition of the droplets comprising the biopolymers to the solid support, the solid support is dried. In some instances, the dried solid support is later resolved. In some instances, the solid support is stored for later use. In some instances, the solid support further comprises an index map of the biopolymers. In some instances, the solid support further comprises metadata.

Devices described herein can comprise power sources used to energize various components of the device. Synthesis components in the solid support are optionally powered by an external power source, or a power source integrated into the solid support. Power sources may comprise batteries, solar cells, thermoelectric generators, inductive (wireless) power units, kinetic energy charger, cellular telephones, tablets, or other power source suitable for use with the synthesis components or devices described herein. In some instances, synthesis components, surfaces, or devices described herein are portable.

Fluids comprising reagents, wash solvents, or other synthesis components are deposited on the synthesis surface. Unused fluid (prior to contact with the synthesis surface) or waste fluid (after contact with the synthesis surface) is in some instances stored in one or more compartments integrated into the solid support. Alternately or in combination, biopolymers are moved in or out of the solid support for external analysis or storage. For example, synthesized biopolymers are cleaved from loci on the solid support in a droplet, the resulting droplet moved externally to the synthesis area of the solid support. The droplet is optionally dried for storage. In some instances, fluids are stored externally from the solid support. In some instances, a device described herein comprises a solid support with a plurality of fluidics ports which allow movement of fluids in and out of the solid support. In some instances, ports are oriented on the sides of the solid support, by other configurations are also suitable for delivery of fluids to the synthesis surface. Such a device often comprises, for example, at least 1, 2, 5, 10, 20, 50, 100, 200, 500, 1000, 2000, 5000, or at least 10,000 ports per mm length of a solid support. In some instances, a device described herein comprises about 100 to about 5000 ports per mm per length of a solid support.

Described herein are devices, compositions, systems and methods for solid support-based biopolymers synthesis and storage, wherein the solid support has varying dimensions. In some instances, a size of the solid support is between about 40 and 120 mm by between about 25 and 100 mm. In some instances, a size of the solid support is about 80 mm by about 50 mm. In some instances, a width of a solid support is at least or about 10 mm, 20 mm, 40 mm, 60 mm, 80 mm, 100 mm, 150 mm, 200 mm, 300 mm, 400 mm, 500 mm, or more than 500 mm. In some instances, a height of a solid support is at least or about 10 mm, 20 mm, 40 mm, 60 mm, 80 mm, 100 mm, 150 mm, 200 mm, 300 mm, 400 mm, 500 mm, or more than 500 mm. In some instances, the solid support has a planar surface area of at least or about 100 mm²; 200 mm²; 500 mm²; 1,000 mm²; 2,000 mm²; 4,500 mm²; 5,000 mm²; 10,000 mm²; 12,000 mm²; 15,000 mm²; 20,000 mm²; 30,000 mm²; 40,000 mm²; 50,000 mm² or more. In some instances, the thickness of the solid support is between about 50 mm and about 2000 mm, between about 50 mm and about 1000 mm, between about 100 mm and about 1000 mm, between about 200 mm and about 1000 mm, or between about 250 mm and about 1000 mm. Non-limiting examples thickness of the solid support include 275 mm, 375 mm, 525 mm, 625 mm, 675 mm, 725 mm, 775 mm and 925 mm. In some instances, the thickness of the solid support is at least or about 0.5 mm, 1.0 mm, 1.5 mm, 2.0 mm, 2.5 mm, 3.0 mm, 3.5 mm, 4.0 mm, or more than 4.0 mm.

Described herein are devices wherein two or more solid supports are assembled. In some instances, solid supports are interfaced together on a larger unit. Interfacing may comprise exchange of fluids, electrical signals, or other medium of exchange between solid supports. This unit is capable of interface with any number of servers, computers, or networked devices. For example, a plurality of solid support is integrated onto a rack unit, which is conveniently inserted or removed from a server rack. The rack unit may comprise any number of solid supports. In some instances, the rack unit comprises at least 1, 2, 5, 10, 20, 50, 100, 200, 500, 1000, 2000, 5000, 10,000, 20,000, 50,000, 100,000 or more than 100,000 solid supports.

Access to biopolymer (e.g., nucleic acid) information in some instances is achieved by cleavage of biopolymers from all or a portion of a solid support. Cleavage in some instances comprises exposure to chemical reagents (ammonia or other reagent), electrical potential, radiation, heat, light, acoustics, or other form of energy capable of manipulating chemical bonds. In some instances, cleavage occurs by charging one or more electrodes in the vicinity of the polynucleotides. In some instances, electromagnetic radiation in the form of UV light is used for cleavage of biopolymers such as polynucleotides. In some instances, a lamp is used for cleavage of biopolymers, and a mask mediates exposure locations of the UV light to the surface. In some instances, a laser is used for cleavage of biopolymers, and a shutter opened/closed state controls exposure of the UV light to the surface. In some instances, access to biopolymer (e.g., nucleic acid) information (including removal/addition of racks, solid supports, reagents, nucleic acids, or other component) is completely automated.

Solid supports as described herein comprise an active area. In some instances, the active area comprises addressable regions or loci for biopolymer synthesis. In some instances, the active area comprises addressable regions or loci for biopolymer storage.

The active area comprises varying dimensions. For example, the dimension of the active area is between about 1 mm to about 50 mm by about 1 mm to about 50 mm. In some instances, the active area comprises a width of at least or about 0.5 mm, 1 mm, 1.5 mm, 2 mm, 2.5 mm, 3 mm, 5 mm, 7 mm, 10 mm, 12 mm, 14 mm, 16 mm, 18 mm, 20 mm, 25 mm, 30 mm, 35 mm, 40 mm, 45 mm, 50 mm, 60 mm, 70 mm, 80 mm, or more than 80 mm. In some instances, the active area comprises a height of at least or about 0.5 mm, 1 mm, 1.5 mm, 2 mm, 2.5 mm, 3 mm, 5 mm, 7 mm, 10 mm, 12 mm, 14 mm, 16 mm, 18 mm, 20 mm, 25 mm, 30 mm, 35 mm, 40 mm, 45 mm, 50 mm, 60 mm, 70 mm, 80 mm, or more than 80 mm.

Described herein are devices, systems, and methods for solid support-based biopolymer synthesis and storage, wherein the solid support has a number of sites (e.g., spots) or positions for synthesis or storage. In some instances, the solid support comprises up to or about 10,000 by 10,000 positions in an area. In some instances, the solid support comprises between about 1000 and 20,000 by between about 1000 and 20,000 positions in an area. In some instances, the solid support comprises at least or about 10, 30, 50, 75, 100, 200, 300, 400, 500, 1000, 2000, 3000, 4000, 5000, 6000, 7000, 8000, 9000, 10,000, 12,000, 14,000, 16,000, 18,000, 20,000 positions by least or about 10, 30, 50, 75, 100, 200, 300, 400, 500, 1000, 2000, 3000, 4000, 5000, 6000, 7000, 8000, 9000, 10,000, 12,000, 14,000, 16,000, 18,000, 20,000 positions in an area. In some instances, the area is up to 0.25, 0.5, 0.75, 1.0, 1.25, 1.5, or 2.0 inches squared.

In some instances, the solid support comprises addressable loci having a pitch of at least or about 0.1 μm, 0.2 μm, 0.25 μm, 0.3 μm, 0.4 μm, 0.5 μm, 1.0 μm, 1.5 μm, 2.0 μm, 2.5 μm, 3.0 μm, 3.5 μm, 4.0 μm, 4.5 μm, 5 μm, 6 μm, 7 μm, 8 μm, 9 μm, 10 μm, or more than 10 μm. In some instances, the solid support comprises addressable loci having a pitch of about 5 μm. In some instances, the solid support comprises addressable loci having a pitch of about 2 μm. In some instances, the solid support comprises addressable loci having a pitch of about 1 μm. In some instances, the solid support comprises addressable loci having a pitch of about 0.2 μm. In some instances, the solid support comprises addressable loci having a pitch of about 0.2 μm to about 10 μm, about 0.2 μm to about 8 μm, about 0.5 μm to about 10 μm, about 1 μm to about 10 μm, about 2 μm to about 8 μm, about 3 μm to about 5 μm, about 1 μm to about 3 μm or about 0.5 μm to about 3 μm. In some instances, the solid support comprises addressable loci having a pitch of about 0.1 μm to about 3 μm. In some instances, the solid support comprises addressable loci having a pitch of less than 0.5 μm.

The solid support for biopolymer synthesis or storage as described herein comprises a high capacity for storage of data. For example, the capacity of the solid support is at least or about 1, 2, 3, 4, 5, 6, 7, 8, 9, 10, 20, 50, 100, 200, 300, 400, 500, 600, 700, 800, 900, 1000, or more than 1000 petabytes. In some instances, the capacity of the solid support is between about 1 to about 10 petabytes or between about 1 to about 100 petabytes. In some instances, the capacity of the solid support is about 100 petabytes. In some instances, the data is stored as addressable arrays of packets as droplets. In some instances, the data is stored as addressable arrays of packets as droplets on a spot. In some instances, the data is stored as addressable arrays of packets as dry wells. In some instances, the addressable arrays comprise at least or about 1, 2, 3, 4, 5, 6, 7, 8, 9, 10, 20, 50, 100, 200, or more than 200 gigabytes of data. In some instances, the addressable arrays comprise at least or about 1, 2, 3, 4, 5, 6, 7, 8, 9, 10, 20, 50, 100, 200, or more than 200 terabytes of data. In some instances, an item of information is stored in a background of data. For example, an item of information encodes for about 10 to about 100 megabytes of data and is stored in 1 petabyte of background data. In some instances, an item of information encodes for at least or about 1, 10, 20, 30, 40, 50, 60, 70, 80, 90, 100, 150, 200, 300, 400, 500, or more than 500 megabytes of data and is stored in 1, 10, 20, 30, 40, 50, 60, 70, 80, 90, 100, 150, 200, 300, 400, 500, or more than 500 petabytes of background data.

Provided herein are devices, systems, and methods for solid support-based biopolymer synthesis and storage, wherein following synthesis, the biopolymers are collected in packets as one or more droplets. In some instances, the biopolymer is a polynucleotide, which is collected in packets as one or more droplets and stored. In some instances, a number of droplets is at least or about 1, 10, 20, 50, 100, 200, 300, 500, 1000, 2500, 5000, 75000, 10,000, 25,000, 50,000, 75,000, 100,000, 1 million, 5 million, 10 million, 25 million, 50 million, 75 million, 100 million, 250 million, 500 million, 750 million, or more than 750 million droplets. In some instances, a droplet volume comprises 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, or more than 100 μm (micrometer) in diameter. In some instances, a droplet volume comprises 1 μm-100 μm, 10 μm-90 μm, 20 μm-80 μm, 30 μm-70 μm, or 40 μm-50 μm in diameter.

In some instances, the biopolymers that are collected in the packets comprise a similar sequence. In some instances, the biopolymers further comprise a non-identical sequence to be used as a tag or barcode. For example, the non-identical sequence is used to index the biopolymers stored on the solid support and to later search for specific biopolymer-based on the non-identical sequence. Exemplary tag or barcode lengths include barcode sequences comprising, without limitation, about 1, 2, 3, 4, 5, 6, 7, 8, 9, 10, 15, 20, 25 or more bases in length. In some instances, the tag or barcode comprise at least or about 10, 50, 75, 100, 200, 300, 400, or more than 400 base pairs in length.

Provided herein are devices, systems, and methods for solid support-based biopolymer synthesis and storage, wherein the biopolymers are collected in packets comprising redundancy. For example, the packets comprise about 100 to about 1000 copies of each polynucleotide. In some instances, the packets comprise at least or about 50, 75, 100, 200, 300, 400, 500, 600, 700, 800, 900, 1000, 1200, 1400, 1600, 1800, 2000, or more than 2000 copies of each polynucleotide. In some instances, the packets comprise about 1000× to about 5000× synthesis redundancy. Synthesis redundancy in some instances is at least or about 500×, 1000×, 1500×, 2000×, 2500×, 3000×, 3500×, 4000×, 5000×, 6000×, 7000×, 8000×, or more than 8000×. The biopolymers (e.g., polynucleotides) that are synthesized using solid support-based methods as described herein comprise various lengths. In some instances, the biopolymer is a polynucleotide that is synthesized and further stored on the solid support. In some instances, the biopolymer length is in between about 100 to about 1000 bases. In some instances, the biopolymers comprise at least or about 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, 2000, or more than 2000 bases in length.

Biopolymer-Based Information Storage

Provided herein are devices, compositions, systems and methods for biopolymer-based information (data) storage. In some instances, the biopolymer is a nucleic acid. In some instances, the biopolymer is a peptide. In a first step, a digital sequence encoding an item of information (i.e., digital information in a binary code for processing by a computer) is received. An encryption scheme is applied to convert the digital sequence from a binary code to a nucleic acid sequence. A surface material for nucleic acid extension, a design for loci for biopolymer (e.g., nucleic acid) extension (aka, arrangement spots), and reagents for biopolymer synthesis are selected. The surface of a structure is prepared for biopolymer synthesis. De novo biopolymer synthesis is performed. The synthesized biopolymers are stored and available for subsequent release, in whole or in part. Once released, the biopolymers, in whole or in part, are sequenced, subject to decryption to convert nucleic sequence back to digital sequence. The digital sequence is then assembled to obtain an alignment encoding for the original item of information.

Optionally, an early step of data storage process disclosed herein includes obtaining or receiving one or more items of information in the form of an initial code. Items of information include, without limitation, text, audio and visual information. Exemplary sources for items of information include, without limitation, books, periodicals, electronic databases, medical records, letters, forms, voice recordings, animal recordings, biological profiles, broadcasts, films, short videos, emails, bookkeeping phone logs, internet activity logs, drawings, paintings, prints, photographs, pixelated graphics, and software code. Exemplary biological profile sources for items of information include, without limitation, gene libraries, genomes, gene expression data, and protein activity data. Exemplary formats for items of information include, without limitation, .txt, .PDF, .doc, .docx, .ppt, .pptx, .xls, .xlsx, .rtf, .jpg, .gif, .psd, .bmp, .tiff, .png, and .mpeg. The amount of individual file sizes encoding for an item of information, or a plurality of files encoding for items of information, in digital format include, without limitation, up to 1024 bytes (equal to 1 KB), 1024 KB (equal to 1 MB), 1024 MB (equal to 1 GB), 1024 GB (equal to 1 TB), 1024 TB (equal to 1PB), 1 exabyte, 1 zettabyte, 1 yottabyte, 1 xenottabyte or more. In some instances, an amount of digital information is at least 1 gigabyte (GB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 gigabytes. In some instances, the amount of digital information is at least 1 terabyte (TB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 terabytes. In some instances, the amount of digital information is at least 1 petabyte (PB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 petabytes.

### Structures for Biopolymer Synthesis

Provided herein are rigid or flexibles structures for biopolymer synthesis. In some instances, the biopolymer is a polynucleotide. In the case of rigid structures, provided herein are devices having a structure for the generation of a library of polynucleotides. In some instances, the structure comprises a plate. An exemplary structure has about the same size dimensions as a standard 96 well plate: 140 mm by 90 mm. The structure comprises clusters grouped in 24 regions or sub-fields, each sub-field comprising an array of 256 clusters. A single cluster can have a Y axis cluster pitch (distance from center to center of adjacent clusters) of 1079.210 um or 1142.694 um, and an X axis cluster pitch of 1125 um. An illustrative cluster has a Y axis loci pitch (distance from center to center of adjacent loci) of 63.483 um, and an X axis loci pitch of 75 um. The locus width at the longest part, e.g., diameter for a circular locus, can be 50 um, and the distance between loci can be 24 um. The loci may be flat, wells, or channels. An exemplary channel arrangement has a plate comprising a main channel and a plurality of channels connected to the main channel. The connection between the main channel and the plurality of channels provides for a fluid communication for flow paths from the main channel to the each of the plurality of channels. A plate described herein can comprise multiple main channels. The plurality of channels collectively forms a cluster within the main channel.

In the case of flexible structures, provided herein are devices wherein the flexible structure comprises a continuous loop wrapped around one or more fixed structures, e.g., a pair of rollers or a non-continuous flexible structure wrapped around separate fixed structures, e.g., a pair reels. In some instances, the structures comprise multiple regions for biopolymer synthesis. An exemplary structure has a plate comprising distinct regions for biopolymer synthesis. The distinct regions may be separated by breaking or cutting. Each of the distinct regions may be further released, sequenced, decrypted, and read or stored. An alternative structure has a tape comprising distinct regions for biopolymer synthesis. The distinct regions may be separated by breaking or cutting. Each of the distinct regions may be further released, sequenced, decrypted, and read or stored. Provided herein are flexible structures having a surface with a plurality of loci for biopolymer extension. Each locus in a portion of the flexible structure, may be a substantially planar spot (e.g., flat), a channel, or a well. In some instances, each locus of the structure has a width of about 10 um and a distance between the center of each structure of about 21 um. Loci may comprise, without limitation, circular, rectangular, tapered, or rounded shapes. Alternatively, or in combination, the structures are rigid. In some instances, the rigid structures comprise loci for biopolymer synthesis. In some instances, the rigid structures comprise substantially planar regions, channels, or wells for biopolymer synthesis.

In some instances, a well described herein has a width to depth (or height) ratio of 1 to 0.01, wherein the width is a measurement of the width at the narrowest segment of the well. In some instances, a well described herein has a width to depth (or height) ratio of 0.5 to 0.01, wherein the width is a measurement of the width at the narrowest segment of the well. In some instances, a well described herein has a width to depth (or height) ratio of about 0.01, 0.05, 0.1, 0.15, 0.16, 0.2, 0.5, or 1. Provided herein are structures for biopolymer (e.g., polynucleotide) synthesis comprising a plurality of discrete loci for biopolymer synthesis. Exemplary structures for the loci include, without limitation, substantially planar regions, channels, wells or protrusions. Structures described herein are may comprise a plurality of clusters, each cluster comprising a plurality of wells, loci or channels. Alternatively, described herein are may comprise a homogenous arrangement of wells, loci or channels. Structures provided herein may comprise wells having a height or depth from about 5 μm to about 500 pm, from about 5 μm to about 400 pm, from about 5 μm to about 300 pm, from about 5 μm to about 200 pm, from about 5 pm to about 100 pm, from about 5 μm to about 50 pm, or from about 10 μm to about 50 pm. In some instances, the height of a well is less than 100 pm, less than 80 pm, less than 60 pm, less than 40 pm or less than 20 pm. In some instances, well height is about 10 pm, 20 pm, 30 pm, 40 μm, 50 μm, 60 μm, 70 μm, 80 μm, 90 μm, 100 μm, 200 μm, 300 μm, 400 μm, 500 μm or more. In some instances, the height or depth of the well is at least 10 nm, 25 nm, 50 nm, 75 nm, 100 nm, 200 nm, 300 nm, 400 nm, 500 nm, 600 nm, 700 nm, 800 nm, 900 nm, 1000 nm, or more than 1000 nm. In some instances, the height or depth of the well is in a range of about 10 nm to about 1000 nm, about 25 nm to about 900 nm, about 50 nm to about 800 nm, about 75 nm to about 700 nm, about 100 nm to about 600 nm, or about 200 nm to about 500. In some instances, the height or depth of the well is in a range of about 50 nm to about 1 um. In some instances, well height is about 10 nm, 20 nm, 30 nm, 40 nm, 50 nm, 60 nm, 70 nm, 80 nm, 90 nm, 100 nm, 200 nm, 300 nm, 400 nm, 500 nm, 700 nm, 800 nm, 900 nm, or about 1000 nm.

Structures for biopolymer (e.g., polynucleotide) synthesis provided herein may comprise channels. The channels may have a width to depth (or height) ratio of 1 to 0.01, wherein the width is a measurement of the width at the narrowest segment of the microchannel. In some instances, a channel described herein has a width to depth (or height) ratio of 0.5 to 0.01, wherein the width is a measurement of the width at the narrowest segment of the microchannel. In some instances, a channel described herein has a width to depth (or height) ratio of about 0.01, 0.05, 0.1, 0.15, 0.16, 0.2, 0.5, or 1.

Described herein are structures for biopolymer synthesis comprising a plurality of discrete loci. Structures comprise, without limitation, substantially planar regions, channels, protrusions, or wells for biopolymer synthesis. In some instances, structures described herein are provided comprising a plurality of channels, wherein the height or depth of the channel is from about 5 μm to about 500 μm, from about 5 μm to about 400 μm, from about 5 μm to about 300 μm, from about 5 μm to about 200 μm, from about 5 μm to about 100 μm, from about 5 μm to about 50 μm, or from about 10 μm to about 50 μm. In some cases, the height of a channel is less than 100 μm, less than 80 μm, less than 60 μm, less than 40 μm or less than 20 μm. In some cases, channel height is about 10 μm, 20 μm, 30 μm, 40 μm, 50 μm, 60 μm, 70 μm, 80 μm, 90 μm, 100 μm, 200 μm, 300 μm, 400 μm, 500 μm or more. In some instances, the height or depth of the channel is at least 10 nm, 25 nm, 50 nm, 75 nm, 100 nm, 200 nm, 300 nm, 400 nm, 500 nm, 600 nm, 700 nm, 800 nm, 900 nm, 1000, or more than 1000 nm. In some instances, the height or depth of the channel is in a range of about 10 nm to about 1000 nm, about 25 nm to about 900 nm, about 50 nm to about 800 nm, about 75 nm to about 700 nm, about 100 nm to about 600 nm, or about 200 nm to about 500. Channels described herein may be arranged on a surface in clusters or as a homogenous field.

The width of a locus on the surface of a structure for biopolymer synthesis described herein may be from about 0.1 μm to about 500 μm, from about 0.5 μm to about 500 μm, from about 1 μm to about 200 μm, from about 1 μm to about 100 μm, from about 5 μm to about 100 μm, or from about 0.1 μm to about 100 μm, for example, about 90 μm, 80 μm, 70 μm, 60 μm, 50 μm, 40 μm, 30 μm, 20 μm, 10 μm, 5 μm, 1 μm or 0.5 μm. In some instances, the width of a locus is less than about 100 μm, 90 μm, 80 μm, 70 μm, 60 μm, 50 μm, 40 μm, 30 μm, 20 μm or 10 μm. In some instances, the width of a locus is at least 10 nm, 25 nm, 50 nm, 75 nm, 100 nm, 200 nm, 300 nm, 400 nm, 500 nm, 600 nm, 700 nm, 800 nm, 900 nm, 1000 nm, or more than 1000 nm. In some instances, the width of a locus is in a range of about 10 nm to about 1000 nm, about 25 nm to about 900 nm, about 50 nm to about 800 nm, about 75 nm to about 700 nm, about 100 nm to about 600 nm, or about 200 nm to about 500. In some instances, the width of a locus is in a range of about 50 nm to about 1000 nm. In some instances, the distance between the center of two adjacent loci is from about 0.1 μm to about 500 μm, 0.5 μm to about 500 μm, from about 1 μm to about 200 μm, from about 1 μm to about 100 μm, from about 5 μm to about 200 μm, from about 5 μm to about 100 μm, from about 5 μm to about 50 μm, or from about 5 μm to about 30 μm, for example, about 20 μm. In some instances, the total width of a locus is about 5 μm, 10 μm, 20 μm, 30 μm, 40 μm, 50 μm, 60 μm, 70 μm, 80 μm, 90 μm, or 100 μm. In some instances, the total width of a locus is about 1 μm to 100 μm, 30 μm to 100 μm, or 50 μm to 70 μm. In some instances, the distance between the center of two adjacent loci is from about 0.5 μm to about 2 μm, 0.5 μm to about 2 μm, from about 0.75 μm to about 2 μm, from about 1 μm to about 2 μm, from about 0.2 μm to about 1 μm, from about 0.5 μm to about 1.5 μm, from about 0.5 μm to about 0.8 μm, or from about 0.5 μm to about 1 μm, for example, about 1 μm. In some instances, the total width of a locus is about 50 nm, 0.1 μm, 0.2 μm, 0.3 μm, 0.4 μm, 0.5 μm, 0.6 μm, 0.7 μm, 0.8 μm, 0.9 μm, 1 μm, 1.1 μm, 1.2 μm, 1.3 μm, 1.4 μm, or 1.5 μm. In some instances, the total width of a locus is about 0.5 μm to 2 μm, 0.75 μm to 1 μm, or 0.9 μm to 2 μm.

In some instances, each locus supports the synthesis of a population of biopolymers having a different sequence than a population of polynucleotides grown on another locus. Provided herein are surfaces which comprise at least 10, 100, 256, 500, 1000, 2000, 3000, 4000, 5000, 6000, 7000, 8000, 9000, 10000, 11000, 12000, 13000, 14000, 15000, 20000, 30000, 40000, 50000 or more clusters. Provided herein are surfaces which comprise more than 2,000; 5,000; 10,000; 20,000; 30,000; 50,000; 100,000; 200,000; 300,000; 400,000; 500,000; 600,000; 700,000; 800,000; 900,000; 1,000,000; 5,000,000; or 10,000,000 or more distinct loci. In some cases, each cluster includes 1, 2, 3, 4, 5, 6, 7, 8, 9, 10, 20, 30, 40, 50, 60, 70, 80, 90, 100, 120, 130, 150, 200, 500 or more loci. In some cases, each cluster includes 50 to 500, 50 to 200, 50 to 150, or 100 to 150 loci. In some cases, each cluster includes 100 to 150 loci. In some instances, each cluster includes 109, 121, 130 or 137 loci.

Provided herein are loci having a width at the longest segment of 5 μm to 100 μm. In some cases, the loci have a width at the longest segment of about 30 μm, 35 μm, 40 μm, 45 μm, 50 μm, 55 μm, or 60 μm. In some cases, the loci are channels having multiple segments, wherein each segment has a center to center distance apart of 5 μm to 50 μm. In some cases, the center to center distance apart for each segment is about 5 μm, 10 μm, 15 μm, 20 μm, or 25 μm.

In some instances, the number of distinct biopolymers synthesized on the surface of a structure described herein is dependent on the number of distinct loci available in the substrate. In some instances, the density of loci within a cluster of a substrate is at least or about 1 locus per mm², 10 loci per mm², 25 loci per mm², 50 loci per mm², 65 loci per mm², 75 loci per mm², 100 loci per mm², 130 loci per mm², 150 loci per mm², 175 loci per mm², 200 loci per mm², 300 loci per mm², 400 loci per mm², 500 loci per mm², 1,000 loci per mm², 10⁴ loci per mm², 10⁵ loci per mm², 10⁶ loci per mm², or more. In some cases, a substrate comprises from about 10 loci per mm² to about 500 mm², from about 25 loci per mm² to about 400 mm², from about 50 loci per mm² to about 500 mm², from about 100 loci per mm² to about 500 mm², from about 150 loci per mm² to about 500 mm², from about 10 loci per mm² to about 250 mm², from about 50 loci per mm² to about 250 mm², from about 10 loci per mm² to about 200 mm², or from about 50 loci per mm² to about 200 mm². In some cases, a substrate comprises from about 10⁴ loci per mm² to about 10⁵ mm². In some cases, a substrate comprises from about 10⁵ loci per mm² to about 10′ mm². In some cases, a substrate comprises at least 10⁵ loci per mm². In some cases, a substrate comprises at least 10⁶ loci per mm². In some cases, a substrate comprises at least 10⁷ loci per mm². In some cases, a substrate comprises from about 10⁴ loci per mm² to about 10⁵ mm².

In some instances, the density of loci within a cluster of a substrate is at least or about 1 locus per μm², 10 loci per μm², 25 loci per μm², 50 loci per μm², 65 loci per μm², 75 loci per μm², 100 loci per μm², 130 loci per μm², 150 loci per μm², 175 loci per μm², 200 loci per μm², 300 loci per μm², 400 loci per μm², 500 loci per μm², 1,000 loci per μm² or more. In some cases, a substrate comprises from about 10 loci per μm² to about 500 μm², from about 25 loci per μm² to about 400 μm², from about 50 loci per μm² to about 500 μm², from about 100 loci per μm² to about 500 μm², from about 150 loci per μm² to about 500 μm², from about 10 loci per μm² to about 250 μm², from about 50 loci per μm² to about 250 μm², from about 10 loci per μm² to about 200 μm², or from about 50 loci per μm² to about 200 μm².

In some instances, the distance between the centers of two adjacent loci within a cluster is from about 10 μm to about 500 μm, from about 10 μm to about 200 μm, or from about 10 μm to about 100 μm. In some cases, the distance between two centers of adjacent loci is greater than about 10 μm, 20 μm, 30 μm, 40 μm, 50 μm, 60 μm, 70 μm, 80 μm, 90 μm or 100 μm. In some cases, the distance between the centers of two adjacent loci is less than about 200 μm, 150 μm, 100 μm, 80 μm, 70 μm, 60 μm, 50 μm, 40 μm, 30 μm, 20 μm or 10 μm. In some cases, the distance between the centers of two adjacent loci is less than about 10000 nm, 8000 nm, 6000 nm, 4000 nm, 2000 nm 1000 nm, 800 nm, 600 nm, 400 nm, 200 nm, 150 nm, 100 nm, 80 nm, 70 nm, 60 nm, 50 nm, 40 nm, 30 nm, 20 nm or 10 nm. In some instances, each square meter of a structure described herein allows for at least 10⁷, 10⁸, 10⁹, 10¹⁰, 10¹¹ loci, where each locus supports one polynucleotide. In some instances, 10⁹ polynucleotides are supported on less than about 6, 5, 4, 3, 2 or 1 m² of a structure described herein.

In some instances, a structure described herein provides support for the synthesis of more than 2,000; 5,000; 10,000; 20,000; 30,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical biopolymers (e.g., polynucleotides). In some cases, the structure provides support for the synthesis of more than 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more biopolymers encoding for distinct sequences. In some instances, at least a portion of the biopolymers have an identical sequence or are configured to be synthesized with an identical sequence. In some instances, the structure provides a surface environment for the growth of biopolymers having at least 50, 60, 70, 75, 80, 85, 90, 95, 100, 110, 120, 130, 140, 150, 160, 175, 200, 225, 250, 275, 300, 325, 350, 375, 400, 425, 450, 475, 500 bases or more. In some arrangements, structures for biopolymer synthesis described herein comprise sites for biopolymer synthesis in a uniform arrangement.

In some instances, biopolymers are synthesized on distinct loci of a structure, wherein each locus supports the synthesis of a population of biopolymers. In some cases, each locus supports the synthesis of a population of biopolymers having a different sequence than a population of biopolymers grown on another locus. In some instances, the loci of a structure are located within a plurality of clusters. In some instances, a structure comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some instances, a structure comprises more than 2,000; 5,000; 10,000; 100,000; 200,000; 300,000; 400,000; 500,000; 600,000;

700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct loci. In some cases, each cluster includes 1, 2, 3, 4, 5, 6, 7, 8, 9, 10, 20, 30, 40, 50, 60, 70, 80, 90, 100, 120, 130, 150 or more loci. In some instances, each cluster includes 50 to 500, 100 to 150, or 100 to 200 loci. In some instances, each cluster includes 109, 121, 130 or 137 loci. In some instances, each cluster includes 5, 6, 7, 8, 9, 10, 11 or 12 loci. In some instances, biopolymers (e.g., polynucleotides) from distinct loci within one cluster have sequences that, when assembled, encode for a contiguous longer biopolymer (e.g., polynucleotide) of a predetermined sequence.

### Structure Size

In some instances, a structure described herein is about the size of a plate (e.g., chip), for example between about 40 and 120 mm by between about 25 and 100 mm. In some instances, a structure described herein has a diameter less than or equal to about 1000 mm, 500 mm, 450 mm, 400 mm, 300 mm, 250 nm, 200 mm, 150 mm, 100 mm or 50 mm. In some instances, the diameter of a substrate is between about 25 mm and 1000 mm, between about 25 mm and about 800 mm, between about 25 mm and about 600 mm, between about 25 mm and about 500 mm, between about 25 mm and about 400 mm, between about 25 mm and about 300 mm, or between about 25 mm and about 200. Non-limiting examples of substrate size include about 300 mm, 200 mm, 150 mm, 130 mm, 100 mm, 84 mm, 76 mm, 54 mm, 51 mm and 25 mm. In some instances, a substrate has a planar surface area of at least 100 mm²; 200 mm²; 500 mm²; 1,000 mm²; 2,000 mm²; 4,500 mm²; 5,000 mm²; 10,000 mm²; 12,000 mm²; 15,000 mm²; 20,000 mm²; 30,000 mm²; 40,000 mm²; 50,000 mm² or more. In some instances, the thickness is between about 50 mm and about 2000 mm, between about 50 mm and about 1000 mm, between about 100 mm and about 1000 mm, between about 200 mm and about 1000 mm, or between about 250 mm and about 1000 mm. Non-limiting examples thickness include 275 mm, 375 mm, 525 mm, 625 mm, 675 mm, 725 mm, 775 mm and 925 mm. In some instances, the thickness is at least or about 0.5 mm, 1.0 mm, 1.5 mm, 2.0 mm, 2.5 mm, 3.0 mm, 3.5 mm, 4.0 mm, or more than 4.0 mm. In some cases, the thickness of varies with diameter and depends on the composition of the substrate. For example, a structure comprising materials other than silicon may have a different thickness than a silicon structure of the same diameter. Structure thickness may be determined by the mechanical strength of the material used and the structure must be thick enough to support its own weight without cracking during handling. In some instances, a structure is more than about 1, 2, 3, 4, 5, 10, 15, 30, 40, 50 feet in any one dimension.

Materials

Provided herein are devices comprising a surface, wherein the surface is modified to support biopolymer synthesis at predetermined locations and with a resulting low error rate, a low dropout rate, a high yield, and a high polymer representation. In some instances, surfaces of devices for biopolymer synthesis provided herein are fabricated from a variety of materials capable of modification to support a de novo biopolymer synthesis reaction. In some cases, the devices are sufficiently conductive, e.g., are able to form uniform electric fields across all or a portion of the devices. Devices described herein may comprise a flexible material. Exemplary flexible materials include, without limitation, modified nylon, unmodified nylon, nitrocellulose, and polypropylene. Devices described herein may comprise a rigid material. Exemplary rigid materials include, without limitation, glass, fuse silica, silicon, silicon dioxide, silicon nitride, plastics (for example, polytetrafluoroethylene, polypropylene, polystyrene, polycarbonate, and blends thereof, and metals (for example, gold, platinum). Devices disclosed herein may be fabricated from a material comprising silicon, polystyrene, agarose, dextran, cellulosic polymers, polyacrylamides, polydimethylsiloxane (PDMS), glass, or any combination thereof. In some cases, devices disclosed herein are manufactured with a combination of materials listed herein or any other suitable material known in the art.

Devices described herein may comprise material having a range of tensile strength. Exemplary materials having a range of tensile strengths include, but are not limited to, nylon (70 MPa), nitrocellulose (1.5 MPa), polypropylene (40 MPa), silicon (268 MPa), polystyrene (40 MPa), agarose (1-10 MPa), polyacrylamide (1-10 MPa), polydimethylsiloxane (PDMS) (3.9-10.81 MPa). Solid supports described herein can have a tensile strength from 1 to 300, 1 to 40, 1 to 10, 1 to 5, or 3 to 11 MPa. Solid supports described herein can have a tensile strength of about 1, 1.5, 2, 3, 4, 5, 6, 7, 8, 9, 10, 11, 20, 25, 40, 50, 60, 70, 80, 90, 100, 150, 200, 250, 270, or more MPa. In some instances, a device described herein comprises a solid support for polynucleotide synthesis that is in the form of a flexible material capable of being stored in a continuous loop or reel, such as a tape or flexible sheet.

Young's modulus measures the resistance of a material to elastic (recoverable) deformation under load. Exemplary materials having a range of Young's modulus stiffness include, but are not limited to, nylon (3 GPa), nitrocellulose (1.5 GPa), polypropylene (2 GPa), silicon (150 GPa), polystyrene (3 GPa), agarose (1-10 GPa), polyacrylamide (1-10 GPa), polydimethylsiloxane (PDMS) (1-10 GPa). Solid supports described herein can have a Young's moduli from 1 to 500, 1 to 40, 1 to 10, 1 to 5, or 3 to 11 GPa. Solid supports described herein can have a Young's moduli of about 1, 1.5, 2, 3, 4, 5, 6, 7, 8, 9, 10, 11, 20, 25, 40, 50, 60, 70, 80, 90, 100, 150, 200, 250, 400, 500 GPa, or more. As the relationship between flexibility and stiffness are inverse to each other, a flexible material has a low Young's modulus and changes its shape considerably under load. In some instances, a solid support described herein has a surface with a flexibility of at least nylon.

In some cases, devices disclosed herein comprise a silicon dioxide base and a surface layer of silicon oxide. Alternatively, the devices may have a base of silicon oxide. Surface of the devices provided here may be textured, resulting in an increase overall surface area for polynucleotide synthesis. Devices disclosed herein in some instances comprise at least 5%, 10%, 25%, 50%, 80%, 90%, 95%, or 99% silicon. Devices disclosed herein in some instances are fabricated from silicon on insulator (SOI) wafer.

The structure may be fabricated from a variety of materials, suitable for the methods and compositions of the invention described herein. In instances, the materials from which the substrates/solid supports of the comprising the invention are fabricated exhibit a low level of polynucleotide binding. In some situations, material that are transparent to visible and/or UV light can be employed. Materials that are sufficiently conductive, e.g. those that can form uniform electric fields across all, or a portion of the substrates/solids support described herein, can be utilized. In some instances, such materials may be connected to an electric ground. In some cases, the substrate or solid support can be heat conductive or insulated. The materials can be chemical resistant and heat resistant to support chemical or biochemical reactions such as a series of polynucleotide synthesis reactions. For flexible materials, materials of interest can include: nylon, both modified and unmodified, nitrocellulose, polypropylene, and the like.

For rigid materials, specific materials of interest include: glass; fuse silica; silicon, plastics (for example polytetrafluoroethylene, polypropylene, polystyrene, polycarbonate, and blends thereof, and the like); metals (for example, gold, platinum, and the like). The structure can be fabricated from a material selected from the group consisting of silicon, polystyrene, agarose, dextran, cellulosic polymers, polyacrylamides, polydimethylsiloxane (PDMS), and glass. The substrates/solid supports or the microstructures, reactors therein may be manufactured with a combination of materials listed herein or any other suitable material known in the art.

In some instances, a substrate disclosed herein comprises a computer readable material. Computer readable materials include, without limitation, magnetic media, reel-to-reel tape, cartridge tape, cassette tape, flexible disk, paper media, film, microfiche, continuous tape (e.g., a belt) and any media suitable for storing electronic instructions. In some cases, the substrate comprises magnetic reel-to-reel tape or a magnetic belt. In some instances, the substrate comprises a flexible printed circuit board.

The light-emitting layers of the devices and systems described herein can be a light-emitting diode (LED), an organic LED (OLED), a polymer LED (PLED), or a phosphorescent organic light-emitting diode (PHOLED). The light-emitting layers of the devices and systems described herein can also be a backlit LED or use fluorescence resonance energy transfer (FRET) (e.g., quantum dots). The LEDs can comprise indium gallium nitride (InGaN), aluminum gallium indium phosphide (AlGaInP), aluminum gallium arsenide (AlGaAs), or gallium phosphide (GaP). The OLEDs can comprise organometallic chelates (e.g., Alp₃), fluorescent and phosphorescent dyes, or conjugated dendrimers. The PLEDs can comprise electroluminescent conductive polymers, including derivatives of poly(p-phenylene vinylene), polyfluorene, poly(naphthalene vinylene)s, water-soluble polymers, or conjugated poly electrolytes. The PHOLEDs can comprise polymers such as poly(N-vinylcarbazole), iridium complexes (e.g., Ir(mppy)₃), polyhedral oligomeric silsesquioxanes (POSS), or other heavy metal complexes. In some instances, a device disclosed herein is manufactured with a combination of materials listed herein or any other suitable material known in the art. In some instances, the light-emitting layer is a micro-LED comprising gallium nitride (GaN).

The seimconductor layer of the device can comprise elemental semiconductors, II-VI compound semiconductors, III-V compound semiconductors, or IV-IV compound semiconductors. In some instances, the semiconductor layer of the device comprises elemental semiconductors, such as Si or Ge. In some instances, the semiconductor layer of the device comprises II-VI compound semiconductors, such as zinc oxide (ZnO), zinc telluride (ZnTe), and zinc sulphide (ZnS). In some instances, the semiconductor layer of the device comprises III-V compound semiconductors, such as indium-phosphide (InP)-based semiconductors (e.g., InGaAsP), gallium-arsenide (GaAs)-based semiconductors (e.g., GaAs, AlGaAs, GaAsSb, InGaAs), or gallium-nitride-based semiconductors (e.g., GaN, InGaN, AlGaN). In some instances, the semiconductor layer of the device comprises IV-IV compound semiconductors, such as silicon carbide (SiC) or a Si—Ge alloy.

Structures described herein may be transparent to visible and/or UV light. In some instances, structures described herein are sufficiently conductive to form uniform electric fields across all or a portion of a structure. In some instances, structures described herein are heat conductive or insulated. In some instances, the structures are chemical resistant and heat resistant to support a chemical reaction such as a biopolymer synthesis reaction. In some instances, the substrate is magnetic. In some instances, the structures comprise a metal or a metal alloy.

Structures for biopolymer synthesis may be over 1, 2, 5, 10, 30, 50 or more feet long in any dimension. In the case of a flexible structure, the flexible structure is optionally stored in a wound state, e.g., in a reel. In the case of a large rigid structure, e.g., greater than 1 foot in length, the rigid structure can be stored vertically or horizontally.

### Surface Preparation

Provided herein are methods to support the immobilization of a biomolecule on a substrate, where a surface of a structure described herein comprises a material and/or is coated with a material that facilitates a coupling reaction with the biomolecule for attachment. To prepare a structure for biomolecule immobilization, surface modifications may be employed that chemically and/or physically alter the substrate surface by an additive or subtractive process to change one or more chemical and/or physical properties of a substrate surface or a selected site or region of the surface. For example, surface modification involves (1) changing the wetting properties of a surface, (2) functionalizing a surface, i.e. providing, modifying or substituting surface functional groups, (3) defunctionalizing a surface, i.e. removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface. In some instances, the surface of a structure is selectively functionalized to produce two or more distinct areas on a structure, wherein at least one area has a different surface or chemical property that another area of the same structure. Such properties include, without limitation, surface energy, chemical termination, surface concentration of a chemical moiety, and the like.

In some instances, a surface of a structure disclosed herein is modified to comprise one or more actively functionalized surfaces configured to bind to both the surface of the substrate and a biomolecule, thereby supporting a coupling reaction to the surface. In some instances, the surface is also functionalized with a passive material that does not efficiently bind the biomolecule, thereby preventing biomolecule attachment at sites where the passive functionalization agent is bound. In some cases, the surface comprises an active layer only defining distinct loci for biomolecule support.

In some instances, the surface is contacted with a mixture of functionalization groups which are in any different ratio. In some instances, a mixture comprises at least 2, 3, 4, 5 or more different types of functionalization agents. In some cases, the ratio of the at least two types of surface functionalization agents in a mixture is about 1:1, 1:2, 1:5, 1:10, 2:10, 3:10, 4:10, 5:10, 6:10, 7:10, 8:10, 9:10, or any other ratio to achieve a desired surface representation of two groups. In some instances, desired surface tensions, wettability, water contact angles, and/or contact angles for other suitable solvents are achieved by providing a substrate surface with a suitable ratio of functionalization agents. In some cases, the agents in a mixture are chosen from suitable reactive and inert moieties, thus diluting the surface density of reactive groups to a desired level for downstream reactions. In some instances, the mixture of functionalization reagents comprises one or more reagents that bind to a biomolecule and one or more reagents that do not bind to a biomolecule. Therefore, modulation of the reagents allows for the control of the amount of biomolecule binding that occurs at a distinct area of functionalization.

In some instances, a method for substrate functionalization comprises deposition of a silane molecule onto a surface of a substrate. The silane molecule may be deposited on a high energy surface of the substrate. In some instances, the high surface energy region includes a passive functionalization reagent. Methods described herein provide for a silane group to bind the surface, while the rest of the molecule provides a distance from the surface and a free hydroxyl group at the end to which a biomolecule attaches.

In some instances, the silane is an organofunctional alkoxysilane molecule. Non-limiting examples of organofunctional alkoxysilane molecules include dimethylchloro-octodecyl-silane, methyldichloro-octodecyl-silane, trichloro-octodecyl-silane, and trimethyl-octodecyl-silane, triethyl-octodecyl-silane. In some instances, the silane is an amino silane. Examples of amino silanes include, without limitation, 11-acetoxyundecyltriethoxysilane, n-decyltriethoxysilane, (3-aminopropyl)trimethoxysilane, (3-aminopropyl)triethoxysilane, glycidyloxypropyl/trimethoxysilane and N-(3-triethoxysilylpropyl)-4-hydroxybutyramide. In some instances, the silane comprises 11-acetoxyundecyltriethoxysilane, n-decyltriethoxysilane, (3-aminopropyl)trimethoxysilane, (3-aminopropyl)triethoxysilane, glycidyloxypropyl/trimethoxysilane, N-(3-triethoxysilylpropyl)-4-hydroxybutyramide, or any combination thereof. In some instances, an active functionalization agent comprises 11-acetoxyundecyltriethoxysilane. In some instances, an active functionalization agent comprises n-decyltriethoxysilane. In some cases, an active functionalization agent comprises glycidyloxypropyltriethoxysilane (GOPS). In some instances, the silane is a fluorosilane. In some instances, the silane is a hydrocarbon silane. In some cases, the silane is 3-iodo-propyltrimethoxysilane. In some cases, the silane is octylchlorosilane.

In some instances, silanization is performed on a surface through self-assembly with organofunctional alkoxysilane molecules. The organofunctional alkoxysilanes are classified according to their organic functions. Non-limiting examples of siloxane functionalizing reagents include hydroxyalkyl siloxanes (silylate surface, functionalizing with diborane and oxidizing the alcohol by hydrogen peroxide), diol (dihydroxyalkyl) siloxanes (silylate surface, and hydrolyzing to diol), aminoalkyl siloxanes (amines require no intermediate functionalizing step), glycidoxysilanes (3-glycidoxypropyl-dimethyl-ethoxysilane, glycidoxy-trimethoxysilane), mercaptosilanes (3-mercaptopropyl-trimethoxysilane, 3-4 epoxycyclohexyl-ethyltrimethoxysilane or 3-mercaptopropyl-methyl-dimethoxysilane), bicyclohepthenyl-trichlorosilane, butyl-aldehydr-trimethoxysilane, or dimeric secondary aminoalkyl siloxanes. Exemplary hydroxyalkyl siloxanes include allyl trichlorochlorosilane turning into 3-hydroxypropyl, or 7-oct-1-enyl trichlorochlorosilane turning into 8-hydroxyoctyl. The diol (dihydroxyalkyl) siloxanes include glycidyl trimethoxysilane-derived (2,3-dihydroxypropyloxy)propyl (GOPS). The aminoalkyl siloxanes include 3-aminopropyl trimethoxysilane turning into 3-aminopropyl (3-aminopropyl-triethoxysilane, 3-aminopropyl-diethoxy-methylsilane, 3-aminopropyl-dimethyl-ethoxysilane, or 3-aminopropyl-trimethoxysilane). In some cases, the dimeric secondary aminoalkyl siloxanes is bis (3-trimethoxysilylpropyl) amine turning into bis(silyloxylpropyl)amine.

Active functionalization areas may comprise one or more different species of silanes, for example, 1, 2, 3, 4, 5, 6, 7, 8, 9, 10 or more silanes. In some cases, one of the one or more silanes is present in the functionalization composition in an amount greater than another silane. For example, a mixed silane solution having two silanes comprises a 99:1, 98:2, 97:3, 96:4, 95:5, 94:6, 93:7, 92:8, 91:9, 90:10, 89:11, 88:12, 87:13, 86:14, 85:15, 84:16, 83:17, 82:18, 81:19, 80:20, 75:25, 70:30, 65:35, 60:40, 55:45 ratio of one silane to another silane. In some instances, an active functionalization agent comprises 11-acetoxyundecyltriethoxysilane and n-decyltriethoxysilane. In some instances, an active functionalization agent comprises 11-acetoxyundecyltriethoxysilane and n-decyltriethoxysilane in a ratio from about 20:80 to about 1:99, or about 10:90 to about 2:98, or about 5:95.

In some instances, functionalization comprises deposition of a functionalization agent to a structure by any deposition technique, including, but not limiting to, chemical vapor deposition (CVD), atomic layer deposition (ALD), plasma enhanced CVD (PECVD), plasma enhanced ALD (PEALD), metal organic CVD (MOCVD), hot wire CVD (HWCVD), initiated CVD (iCVD), modified CVD (MCVD), vapor axial deposition (VAD), outside vapor deposition (OVD), physical vapor deposition (e.g., sputter deposition, evaporative deposition), and molecular layer deposition (MLD).

Any step or component in the following functionalization process be omitted or changed in accordance with properties desired of the final functionalized substrate. In some cases, additional components and/or process steps are added to the process workflows embodied herein. In some instances, a substrate is first cleaned, for example, using a piranha solution. An example of a cleaning process includes soaking a substrate in a piranha solution (e.g., 90% H₂SO₄, 10% H₂O₂) at an elevated temperature (e.g., 120° C.) and washing (e.g., water) and drying the substrate (e.g., nitrogen gas). The process optionally includes a post piranha treatment comprising soaking the piranha treated substrate in a basic solution (e.g., NH₄OH) followed by an aqueous wash (e.g., water). In some instances, a surface of a structure is plasma cleaned, optionally following the piranha soak and optional post piranha treatment. An example of a plasma cleaning process comprises an oxygen plasma etch. In some instances, the surface is deposited with an active functionalization agent following by vaporization. In some instances, the substrate is actively functionalized prior to cleaning, for example, by piranha treatment and/or plasma cleaning.

The process for surface functionalization optionally comprises a resist coat and a resist strip. In some instances, following active surface functionalization, the substrate is spin coated with a resist, for example, SPR™ 3612 positive photoresist. The process for surface functionalization, in various instances, comprises lithography with patterned functionalization. In some instances, photolithography is performed following resist coating. In some instances, after lithography, the surface is visually inspected for lithography defects. The process for surface functionalization, in some instances, comprises a cleaning step, whereby residues of the substrate are removed, for example, by plasma cleaning or etching. In some instances, the plasma cleaning step is performed at some step after the lithography step.

In some instances, a surface coated with a resist is treated to remove the resist, for example, after functionalization and/or after lithography. In some cases, the resist is removed with a solvent, for example, with a stripping solution comprising N-methyl-2-pyrrolidone. In some cases, resist stripping comprises sonication or ultrasonication. In some instances, a resist is coated and stripped, followed by active functionalization of the exposed areas to create a desired differential functionalization pattern.

In some instances, the methods and compositions described herein relate to the application of photoresist for the generation of modified surface properties in selective areas, wherein the application of the photoresist relies on the fluidic properties of the surface defining the spatial distribution of the photoresist. Without being bound by theory, surface tension effects related to the applied fluid may define the flow of the photoresist. For example, surface tension and/or capillary action effects may facilitate drawing of the photoresist into small structures in a controlled fashion before the resist solvents evaporate. In some instances, resist contact points are pinned by sharp edges, thereby controlling the advance of the fluid. The underlying structures may be designed-based on the desired flow patterns that are used to apply photoresist during the manufacturing and functionalization processes. A solid organic layer left behind after solvents evaporate may be used to pursue the subsequent steps of the manufacturing process. Structures may be designed to control the flow of fluids by facilitating or inhibiting wicking effects into neighboring fluidic paths. For example, a structure is designed to avoid overlap between top and bottom edges, which facilitates the keeping of the fluid in top structures allowing for a particular disposition of the resist. In an alternative example, the top and bottom edges overlap, leading to the wicking of the applied fluid into bottom structures. Appropriate designs may be selected accordingly, depending on the desired application of the resist.

In some instances, a structure described herein has a surface that comprises a material having thickness of at least or at least 0.1 nm, 0.5 nm, 1 nm, 2 nm, 5 nm, 10 nm or 25 nm that comprises a reactive group capable of binding nucleosides. Exemplary include, without limitation, glass and silicon, such as silicon dioxide and silicon nitride. In some cases, exemplary surfaces include nylon and PMMA.

In some instances, electromagnetic radiation in the form of UV light is used for surface patterning. In some instances, a lamp is used for surface patterning, and a mask mediates exposure locations of the UV light to the surface. In some instances, a laser is used for surface patterning, and a shutter opened/closed state controls exposure of the UV light to the surface. The laser arrangement may be used in combination with a flexible structure that is capable of moving. In such an arrangement, the coordination of laser exposure and flexible structure movement is used to create patterns of one or more agents having differing nucleoside coupling capabilities.

Described herein are surfaces for polynucleotide synthesis that are reusable. After synthesis and/or cleavage of polynucleotides, a surface may be bathed, washed, cleaned, baked, etched, or otherwise functionally restored to a condition suitable for subsequent polynucleotide synthesis. The number of times a surface is reused and the methods for recycling/preparing the surface for reuse vary depending on subsequent applications. Surfaces prepared for reuse are in some instances reused at least 1, 2, 3, 5, 10, 20, 50, 100, 1,000 or more times. In some instances, the remaining “life” or number of times a surface is suitable for reuse is measured or predicted.

### Material Deposition Systems

In some cases, the synthesized biopolymers are stored on the substrate, for example a solid support. Biopolymer reagents may be deposited on the substrate in a continuous method. Biopolymer reagents may also be deposited on the substrate surface in a non-continuous or drop-on-demand method.

Biopolymer reagents may be deposited on the substrate using a continuous method. Examples of such methods include methods carried out in a flow cell reactor. Biopolymer reagents may also be deposited on the substrate surface in a non-continuous, or drop-on-demand method. Examples of such methods include the electromechanical transfer method, electric thermal transfer method, and electrostatic attraction method. In the electromechanical transfer method, piezoelectric elements deformed by electrical pulses cause the droplets to be ejected. In the electric thermal transfer method, bubbles are generated in a chamber of the device, and the expansive force of the bubbles causes the droplets to be ejected. In the electrostatic attraction method, electrostatic force of attraction is used to eject the droplets onto the substrate. In some cases, the drop frequency is from about 5 KHz to about 500 KHz; from about 5 KHz to about 100 KHz; from about 10 KHz to about 500 KHz; from about 10 KHz to about 100 KHz; or from about 50 KHz to about 500 KHz. In some cases, the frequency is less than about 500 KHz, 200 KHz, 100 KHz, or 50 KHz.

The size of the droplets dispensed correlates to the resolution of the device. In some instances, the devices deposit droplets of reagents at sizes from about 0.01 pl to about 20 pl, from about 0.01 pl to about 10 pl, from about 0.01 pl to about 1 pl, from about 0.01 pl to about 0.5 pl, from about 0.01 pl to about 0.01 pl, or from about 0.05 pl to about 1 pl. In some instances, the droplet size is less than about 1 pl, 0.5 pl, 0.2 pl, 0.1 pl, or 0.05 pl.

In some arrangements, the configuration of a biopolymer synthesis system allows for a continuous biopolymer synthesis process that exploits the flexibility of a substrate for traveling in a reel-to-reel type process. This synthesis process operates in a continuous production line manner with the substrate travelling through various stages of biopolymer synthesis using one or more reels to rotate the position of the substrate. In an exemplary instance, a biopolymer synthesis reaction comprises rolling a substrate: through a solvent bath, beneath a deposition device for phosphoramidite deposition, through a bath of oxidizing agent, through an acetonitrile wash bath, and through the light-directed deblock process. Optionally, the tape is also traversed through a capping bath. A reel-to-reel type process allows for the finished product of a substrate comprising synthesized biopolymers to be easily gathered on a take-up reel, where it can be transported for further processing or storage.

In some arrangements, biopolymer synthesis proceeds in a continuous process as a continuous flexible tape is conveyed along a conveyor belt system. Similar to the reel-to-reel type process, biopolymer synthesis on a continuous tape operates in a production line manner, with the substrate travelling through various stages of biopolymer synthesis during conveyance. However, in a conveyor belt process, the continuous tape revisits a biopolymer synthesis step without rolling and unrolling of the tape, as in a reel-to-reel process. In some arrangements, biopolymer synthesis steps are partitioned into zones and a continuous tape is conveyed through each zone one or more times in a cycle. For example, a polynucleotide synthesis reaction may comprise (1) conveying a substrate through a solvent bath, beneath a deposition device for phosphoramidite deposition, through a bath of oxidizing agent, through an acetonitrile wash bath, and through a light-directed de-block process in a cycle; and then (2) repeating the cycles to achieve synthesized polynucleotides of a predetermined length. After biopolymer synthesis, the flexible substrate is removed from the conveyor belt system and, optionally, rolled for storage. Rolling may be around a reel, for storage. In some instances, a flexible substrate comprising thermoplastic material is coated with monomer coupling reagent. The coating is patterned into loci such that each locus has diameter of about 10 um, with a center-to-center distance between two adjacent loci of about 21 um. In this instance, the locus size is sufficient to accommodate a sessile drop volume of 0.2 pl during a biopolymer synthesis deposition step. In some cases, the locus density is about 2.2 billion loci per m² (1 locus/441×10¹² m²). In some cases, a 4.5 m² substrate comprise about 10 billion loci, each with a 10 um diameter.

In some arrangements, a device for application of one or more reagents to a substrate during a synthesis reaction is configured to deposit reagents and/or nucleoside monomers for nucleoside phosphoramidite-based synthesis. Reagents for polynucleotide synthesis include reagents for polynucleotide extension and wash buffers. As non-limiting examples, the device deposits cleaning reagents, coupling reagents, capping reagents, oxidizers, acetonitrile, gases such as nitrogen gas, and any combination thereof. In addition, the device optionally deposits reagents for the preparation and/or maintenance of substrate integrity. In some instances, the biopolymer synthesizer deposits a drop having a diameter less than about 200 um, 100 um, or 50 um in a volume less than about 1000, 500, 100, 50, or 20 pl. In some cases, the polynucleotide synthesizer deposits between about 1 and 10000, 1 and 5000, 100 and 5000, or 1000 and 5000 droplets per second.

Described herein are devices, methods, systems and compositions where reagents for biopolymer synthesis are recycled or reused. Recycling of reagents may comprise collection, storage, and usage of unused reagents, or purification/transformation of used reagents. For example, a reagent bath is recycled and used for a biopolymer synthesis step on the same or a different surface. Reagents described herein may be recycled 1, 2, 3, 4, 5, 6, 7, 8, 9, 10 or more times. Alternatively, or in combination, a reagent solution comprising a reaction byproduct is filtered to remove the byproduct, and the reagent solution is used for additional biopolymer synthesis reactions.

Many integrated or non-integrated elements are often used with biopolymer synthesis systems. In some instances, a biopolymer synthesis system comprises one or more elements useful for downstream processing of synthesized biopolymers. As an example, the system comprises a temperature control element such as a thermal cycling device. In some instances, the temperature control element is used with a plurality of resolved reactors to perform nucleic acid assembly such as PCA and/or nucleic acid amplification such as PCR.

### De Novo Biopolymer Synthesis

Provided herein are systems and methods for synthesis of a high density of biopolymer on a substrate in a short amount of time. In some instances, the substrate is a flexible substrate. In some instances, the biopolymer is a polynucleotide, and at least 10¹⁰, 10¹¹, 10¹², 10¹³, 10¹⁴, or 10¹⁵ bases are synthesized in one day. In some instances, the biopolymer is a polynucleotide, and at least 10×10⁸, 10×10⁹, 10×10¹⁰, 10×10¹¹, or 10×10¹² polynucleotides are synthesized in one day. In some cases, each polynucleotide synthesized comprises at least 20, 50, 100, 200, 300, 400 or 500 nucleobases. In some cases, these bases are synthesized with a total average error rate of less than about 1 in 100; 200; 300; 400; 500; 1000; 2000; 5000; 10000; 15000; 20000 bases. In some instances, these error rates are for at least 50%, 60%, 70%, 80%, 90%, 95%, 98%, 99%, 99.5%, or more of the polynucleotides synthesized.

In some instances, these at least 90%, 95%, 98%, 99%, 99.5%, or more of the polynucleotides synthesized do not differ from a predetermined sequence for which they encode. In some instances, the error rate for synthesized biopolymers on a substrate using the methods and systems described herein is less than about 1 in 200. In some instances, the error rate for synthesized biopolymers on a substrate using the methods and systems described herein is less than about 1 in 1,000. In some instances, the error rate for synthesized biopolymers on a substrate using the methods and systems described herein is less than about 1 in 2,000. In some instances, the error rate for synthesized biopolymers on a substrate using the methods and systems described herein is less than about 1 in 3,000. In some instances, the error rate for synthesized biopolymers on a substrate using the methods and systems described herein is less than about 1 in 5,000. Individual types of error rates include mismatches, deletions, insertions, and/or substitutions for the polynucleotides synthesized on the substrate. The term “error rate” refers to a comparison of the collective amount of synthesized biopolymer (e.g., polynucleotide) to an aggregate of predetermined biopolymer sequence. In some instances, synthesized biopolymers are polynucleotides disclosed herein, which comprise a tether of 12 to 25 bases. In some instances, the tether comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more bases.

A suitable method for biopolymers (e.g., polynucleotide) synthesis on a substrate of this disclosure is a phosphoramidite method comprising the controlled addition of a phosphoramidite building block, i.e. nucleoside phosphoramidite, to a growing polynucleotide chain in a coupling step that forms a phosphite triester linkage between the phosphoramidite building block and a nucleoside bound to the substrate. In some instances, the nucleoside phosphoramidite is provided to the substrate activated. In some instances, the nucleoside phosphoramidite is provided to the substrate with an activator. In some instances, nucleoside phosphoramidites are provided to the substrate in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition and linkage of a nucleoside phosphoramidite in the coupling step, the substrate is optionally washed. In some instances, the coupling step is repeated one or more additional times, optionally with a wash step between nucleoside phosphoramidite additions to the substrate. In some instances, a polynucleotide synthesis method used herein comprises 1, 2, 3 or more sequential coupling steps.

Prior to coupling, in many cases, the biopolymer (e.g., nucleoside) bound to the substrate is de-protected by removal of a protecting group, where the protecting group functions to prevent polymerization. Protecting groups may comprise any chemical group that prevents extension of the biopolymer chain. The protecting group is removed with electromagnetic radiation such as light. In some instances, the 5′-photolabile protecting group is nitrophenylpropyloxycarbonyl (NPPOC), 2,(3,4-methylenediooxy-6-nitrophenyl)propoxycarbonyl (MNPPOC), benzoyl-NPPOC, or thiophenyl-NPPOC. In some instances, the photolabile protecting group can be an ortho-nitrobenzyl derivative, a coumadin derivative, or another chemical protecting group.

Following coupling, phosphoramidite biopolymer synthesis methods optionally comprise a capping step. In a capping step, the growing biopolymer is treated with a capping agent. A capping step generally serves to block unreacted substrate-bound 5′—OH groups after coupling from further chain elongation, preventing the formation of biopolymers with internal base deletions. Further, phosphoramidites activated with 1H-tetrazole often react, to a small extent, with the O6 position of guanosine.

Following addition of a nucleoside phosphoramidite, and optionally after capping and one or more wash steps, a substrate described herein comprises a bound growing nucleic acid that may be oxidized. The oxidation step comprises oxidizing the phosphite triester into a tetracoordinated phosphate triester, a protected precursor of the naturally occurring phosphate diester internucleoside linkage. In some instances, phosphite triesters are oxidized electrochemically. In some instances, oxidation of the growing biopolymer is achieved by treatment with iodine and water, optionally in the presence of a weak base such as a pyridine, lutidine, or collidine. Oxidation is sometimes carried out under anhydrous conditions using tert-Butyl hydroperoxide or (1S)-(+)-(10-camphorsulfonyl)-oxaziridine (CSO). In some methods, a capping step is performed following oxidation. A second capping step allows for substrate drying, as residual water from oxidation that may persist can inhibit subsequent coupling. Following oxidation, the substrate and growing biopolymer is optionally washed. In some instances, the biopolymer is a polynucleotide, and the step of oxidation is substituted with a sulfurization step to obtain polynucleotide phosphorothioates, wherein any capping steps can be performed after the sulfurization. Many reagents are capable of the efficient sulfur transfer, including, but not limited to, 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

For a subsequent cycle of nucleoside incorporation to occur through coupling, a protected 5′ end (or 3′ end, if synthesis is conducted in a 5′ to 3′ direction) of the substrate bound growing polynucleotide is be removed so that the primary hydroxyl group can react with a next nucleoside phosphoramidite. Methods and compositions described herein provide for controlled deblocking conditions using UV-illumination. In some instances, the substrate bound biopolymer is washed after deblocking. In some cases, efficient washing after deblocking contributes to synthesized biopolymers having a low error rate.

Methods for the synthesis of biopolymers on a substrate described herein may involve an iterating sequence of the following steps: application of a protected monomer to a surface of a substrate feature to link with either the surface, a linker or with a previously deprotected monomer; deprotection of the applied monomer so that it can react with a subsequently applied protected monomer; and application of another protected monomer for linking. One or more intermediate steps include oxidation and/or sulfurization. In some instances, one or more wash steps precede or follow one or all of the steps.

Methods for the synthesis of biopolymers on a substrate described herein may comprise an oxidation step. For example, methods involve an iterating sequence of the following steps: application of a protected monomer to a surface of a substrate feature to link with either the surface, a linker or with a previously deprotected monomer; deprotection of the applied monomer so that it can react with a subsequently applied protected monomer; application of another protected monomer for linking, and oxidation and/or sulfurization. In some instances, one or more wash steps precede or follow one or all of the steps.

Methods for the synthesis of biopolymers on a substrate described herein may further comprise an iterating sequence of the following steps: application of a protected monomer to a surface of a substrate feature to link with either the surface, a linker or with a previously deprotected monomer; deprotection of the applied monomer so that it can react with a subsequently applied protected monomer; and oxidation and/or sulfurization. In some instances, one or more wash steps precede or follow one or all of the steps.

Methods for the synthesis of biopolymers on a substrate described herein may further comprise an iterating sequence of the following steps: application of a protected monomer to a surface of a substrate feature to link with either the surface, a linker or with a previously deprotected monomer; and oxidation and/or sulfurization. In some instances, one or more wash steps precede or follow one or all of the steps. In some instances, the method does not require the oxidation step.

Methods for the synthesis of biopolymers on a substrate described herein may further comprise an iterating sequence of the following steps: application of a protected monomer to a surface of a substrate feature to link with either the surface, a linker or with a previously deprotected monomer; deprotection of the applied monomer so that it can react with a subsequently applied protected monomer; and oxidation and/or sulfurization. In some instances, one or more wash steps precede or follow one or all of the steps. In some instances, the method does not require oxidation.

In some instances, biopolymers are synthesized with photolabile protecting groups, where the hydroxyl groups generated on the surface are blocked by photolabile-protecting groups. When the surface is exposed to UV light, such as through the LED light-emission system of the disclosure, a pattern of free hydroxyl groups on the surface may be generated. These hydroxyl groups can react with photo-protected nucleoside phosphoramidites, according to phosphoramidite chemistry. A second photolithographic mask can be applied, and the surface can be exposed to UV light to generate second pattern of hydroxyl groups, followed by coupling with 5′-photoprotected nucleoside phosphoramidite. Likewise, patterns can be generated, and oligomer chains can be extended. Without being bound by theory, the lability of a photocleavable group depends on the wavelength and polarity of a solvent employed and the rate of photocleavage may be affected by the duration of exposure and the intensity of light. This method can leverage a number of factors such as accuracy in alignment of the LED lights, efficiency of removal of photo-protecting groups, and the yields of the phosphoramidite coupling step. Further, unintended leakage of light into neighboring sites can be minimized. The density of synthesized oligomer per spot can be monitored by adjusting loading of the leader nucleoside on the surface of synthesis.

The surface of a substrate described herein that provides support for biopolymer synthesis may be chemically modified to allow for the synthesized biopolymer chain to be cleaved from the surface. In some instances, the biopolymer chain is cleaved at the same time as the biopolymer is deprotected. In some cases, the biopolymer chain is cleaved after the biopolymer is deprotected. In an exemplary scheme, a trialkoxysilyl amine such as (CH₃CH₂O)₃Si—(CH₂)₂—NH₂ is reacted with surface SiOH groups of a substrate, followed by reaction with succinic anhydride with the amine to create an amide linkage and a free OH on which the nucleic acid chain growth is supported. Cleavage includes gas cleavage with ammonia or methylamine. In some instances, cleavage includes linker cleavage with electrically generated reagents such as acids or bases. In some instances, the biopolymer is a polynucleotide, and once released from the surface, polynucleotides are assembled into larger nucleic acids that are sequenced and decoded to extract stored information.

The surfaces described herein can be reused after biopolymer cleavage to support additional cycles of biopolymer synthesis. For example, the linker can be reused without additional treatment/chemical modifications. In some instances, a linker is non-covalently bound to a substrate surface or a biopolymer. In some embodiments, the linker remains attached to the biopolymer after cleavage from the surface. Linkers in some embodiments comprise reversible covalent bonds such as esters, amides, ketals, beta substituted ketones, heterocycles, or other group that is capable of being reversibly cleaved. Such reversible cleavage reactions are in some instances controlled through the addition or removal of reagents, or by electrochemical processes controlled by electrodes. Optionally, chemical linkers or surface-bound chemical groups are regenerated after a number of cycles, to restore reactivity and remove unwanted side product formation on such linkers or surface-bound chemical groups.

The devices and methods described herein can be used for the enzymatic synthesis of biopolymers. Terminal deoxynucleotidyl transferase (TdT), also known as DNA nucleotidylexotransferase (DNTT) or terminal transferase, is a specialized DNA polymerase expressed in immature, pre-B, or pre-T lymphoid cells and acute lymphoblastic leukemia/lymphoma cells. TdT catalyzes the addition of nucleotides to the 3′-terminus of a DNA molecule. Unlike most DNA polymerases, TdT does not require a template.

The devices and methods disclosed herein can be used with TdT to synthesize polynucleotides, wherein photolabile protecting groups are photocleaved to deprotect reactive hydroxyl groups. In some instances, TdT can be used to add individual bases in controlled de novo synthesis schemes described herein. In some instances, a TdT molecule is conjugated to a single deoxyribonucleoside triphosphate (dNTP) molecule that TdT can incorporate into a primer. After incorporation of the tethered dNTP, the 3′-end of the primer remains covalently bound to TdT and is inaccessible to other TdT-dNTP molecules. Cleaving the linkage between TdT and the incorporated nucleoside releases the primer and allows subsequent extension.

### Assembly

Biopolymers may be designed to collectively span a large region of a predetermined sequence that encodes for information. In some instances, the biopolymer is a polynucleotide. In some instances, larger polynucleotides are generated through ligation reactions to join the synthesized polynucleotides. One example of a ligation reaction is polymerase chain assembly (PCA). In some instances, at least of a portion of the polynucleotides are designed to include an appended region that is a substrate for universal primer binding. For PCA reactions, the pre-synthesized polynucleotides include overlaps with each other (e.g., 4, 20, 40 or more bases with overlapping sequence). During the polymerase cycles, the polynucleotides anneal to complementary fragments and then are filled in by polymerase. Each cycle thus increases the length of various fragments randomly depending on which polynucleotides find each other. Complementarity amongst the fragments allows for forming a complete large span of double-stranded DNA. In some cases, after the PCA reaction is complete, an error correction step is conducted using mismatch repair detecting enzymes to remove mismatches in the sequence. Once larger fragments of a target sequence are generated, they can be amplified. For example, in some cases, a target sequence comprising 5′ and 3′ terminal adapter sequences are amplified in a polymerase chain reaction (PCR) which includes modified primers that hybridize to the adapter sequences. In some cases, the modified primers comprise one or more uracil bases. The use of modified primers allows for removal of the primers through enzymatic reactions centered on targeting the modified base and/or gaps left by enzymes which cleave the modified base pair from the fragment. What remains is a double-stranded amplification product that lacks remnants of adapter sequence. In this way, multiple amplification products can be generated in parallel with the same set of primers to generate different fragments of double-stranded DNA.

Error correction may be performed on synthesized polynucleotides and/or assembled products. An example strategy for error correction involves site-directed mutagenesis by overlap extension PCR to correct errors, which is optionally coupled with two or more rounds of cloning and sequencing. In certain instances, double-stranded nucleic acids with mismatches, bulges and small loops, chemically altered bases and/or other heteroduplexes are selectively removed from populations of correctly synthesized nucleic acids. In some instances, error correction is performed using proteins/enzymes that recognize and bind to or next to mismatched or unpaired bases within double-stranded nucleic acids to create a single or double-strand break or to initiate a strand transfer transposition event. Non-limiting examples of proteins/enzymes for error correction include endonucleases (T7 Endonuclease I, E. coli Endonuclease V, T4 Endonuclease VII, mung bean nuclease, Cell, E. coli Endonuclease IV, UVDE), restriction enzymes, glycosylases, ribonucleases, mismatch repair enzymes, resolvases, helicases, ligases, antibodies specific for mismatches, and their variants. Examples of specific error correction enzymes include T4 endonuclease 7, T7 endonuclease 1, S1, mung bean endonuclease, MutY, MutS, MutH, MutL, cleavase, CELI, and HINF1. In some cases, DNA mismatch-binding protein MutS (Thermus aquaticus) is used to remove failure products from a population of synthesized products. In some instances, error correction is performed using the enzyme Correctase. In some cases, error correction is performed using SURVEYOR endonuclease (Transgenomic), a mismatch-specific DNA endonuclease that scans for known and unknown mutations and polymorphisms for heteroduplex DNA.

### Sequencing

After extraction and/or amplification of biopolymer s from the surface of the structure, suitable sequencing technology may be employed to sequence the biopolymer s. In some cases, the biopolymer is DNA, and the DNA sequence is read on the substrate or within a feature of a structure. In some cases, the polynucleotides stored on the substrate are extracted is optionally assembled into longer nucleic acids and then sequenced.

Biopolymers synthesized and stored on the structures described herein encode data that can be interpreted by reading the sequence of the synthesized polynucleotides and converting the sequence into binary code readable by a computer. In some cases, the sequences require assembly, and the assembly step may need to be at the nucleic acid sequence stage or at the digital sequence stage.

Provided herein are detection systems comprising a device capable of sequencing stored biopolymers, either directly on the structure and/or after removal from the main structure. In cases where the structure is a reel-to-reel tape of flexible material, the detection system comprises a device for holding and advancing the structure through a detection location and a detector disposed proximate the detection location for detecting a signal originated from a section of the tape when the section is at the detection location. In some instances, the signal is indicative of a presence of a polynucleotide. In some instances, the signal is indicative of a sequence of a biopolymer (e.g., a fluorescent signal). In some instances, information encoded within biopolymers on a continuous tape is read by a computer as the tape is conveyed continuously through a detector operably connected to the computer. In some instances, the biopolymer is a polynucleotide, and a detection system comprises a computer system comprising a polynucleotide sequencing device, a database for storage and retrieval of data relating to polynucleotide sequence, software for converting DNA code of a polynucleotide sequence to binary code, a computer for reading the binary code, or any combination thereof.

Provided herein are sequencing systems that can be integrated into the devices described herein. Various methods of sequencing are well known in the art, and comprise “base calling” wherein the identity of a base in the target polynucleotide is identified. In some instances, polynucleotides synthesized using the methods, devices, compositions, and systems described herein are sequenced after cleavage from the synthesis surface. In some instances, sequencing occurs during or simultaneously with polynucleotide synthesis, wherein base calling occurs immediately after or before extension of a nucleoside monomer into the growing polynucleotide chain. Methods for base calling include measurement of electrical currents generated by polymerase-catalyzed addition of bases to a template strand. In some instances, synthesis surfaces comprise enzymes, such as polymerases. In some instances, such enzymes are tethered to electrodes or to the synthesis surface.

Computer Systems

In various aspects, any of the systems described herein are operably linked to a computer and are optionally automated through a computer either locally or remotely. In various instances, the methods and systems of the invention further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the invention. In some instances, the computer systems are programmed to interface between the user specified base sequence and the position of a material deposition device to deliver the correct reagents to specified regions of the substrate.

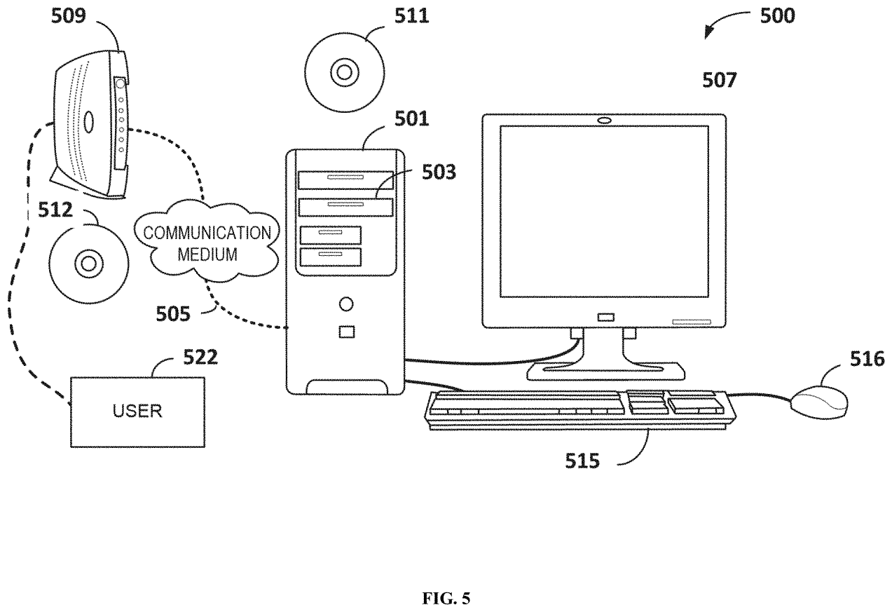

The computer system 500 illustrated in FIG. 5 may be understood as a logical apparatus that can read instructions from media 511 and/or a network port 505, which can optionally be connected to server 509 having fixed media 512. The system can include a CPU 501, disk drives 503, optional input devices such as keyboard 515 and/or mouse 516 and optional monitor 507. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 522.

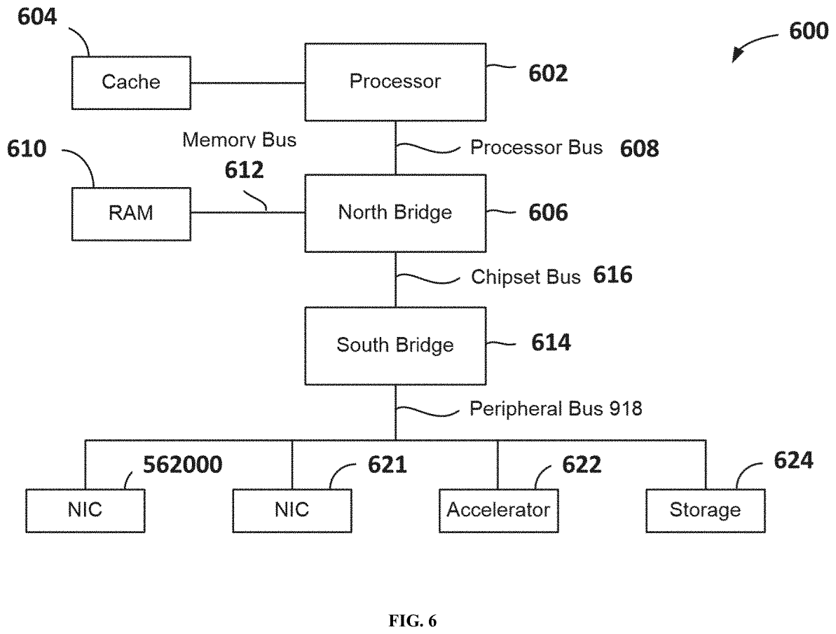

FIG. 6 is a block diagram illustrating a first example architecture of a computer system that can be used in connection with example instances of the present invention. As depicted in FIG. 5, the example computer system can include a processor 602 for processing instructions. Non-limiting examples of processors include: Intel Xeon™ processor, AMD Opteron™ processor, Samsung 32-bit RISC ARM 1176JZ(F)-S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices.

As illustrated in FIG. 6, a high speed cache 604 can be connected to, or incorporated in, the processor 602 to provide a high speed memory for instructions or data that have been recently, or are frequently, used by processor 602. The processor 602 is connected to a north bridge 606 by a processor bus 608. The north bridge 606 is connected to random access memory (RAM) 610 by a memory bus 612 and manages access to the RAM 610 by the processor 602. The north bridge 906 is also connected to a south bridge 614 by a chipset bus 616. The south bridge 614 is, in turn, connected to a peripheral bus 618. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 618. In some alternative architectures, the functionality of the north bridge can be incorporated into the processor instead of using a separate north bridge chip.

In some instances, a system 600 can include an accelerator card 622 attached to the peripheral bus 618. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 624 and can be loaded into RAM 610 and/or cache 604 for use by the processor. The system 600 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example embodiments of the present invention.

In this example, system 600 also includes network interface cards (NICs) 620 and 621 connected to the peripheral bus for providing network interfaces to external storage, such as Network Attached Storage (NAS) and other computer systems that can be used for distributed parallel processing.

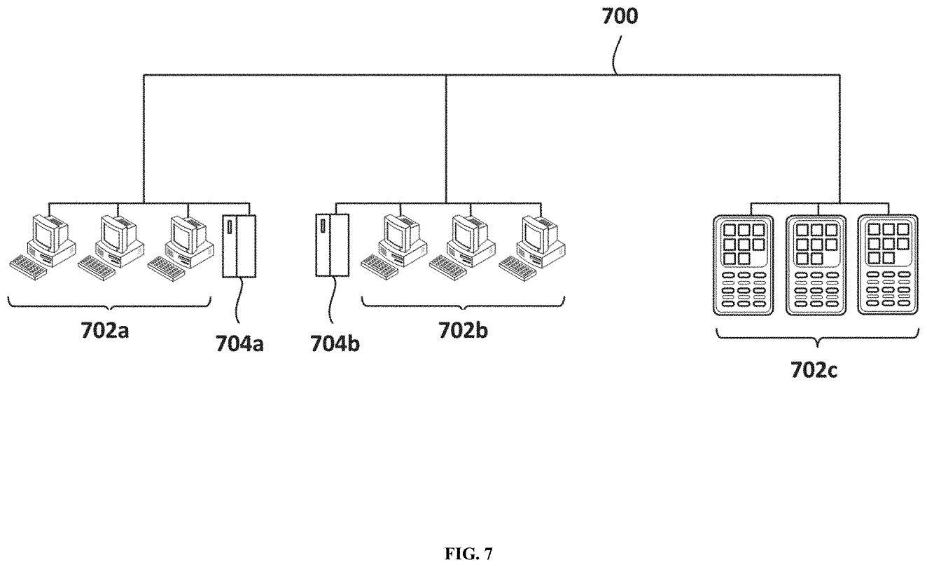

FIG. 7 is a diagram showing a network 700 with a plurality of computer systems 702a, and 702b, a plurality of cell phones and personal data assistants 702c, and Network Attached Storage (NAS) 704a, and 704b. In example embodiments, systems 702a, 702b, and 702c can manage data storage and optimize data access for data stored in Network Attached Storage (NAS) 704a and 704b. A mathematical model can be used for the data and be evaluated using distributed parallel processing across computer systems 702a, and 702b, and cell phone and personal data assistant systems 702c. Computer systems 702a, and 702b, and cell phone and personal data assistant systems 702c can also provide parallel processing for adaptive data restructuring of the data stored in Network Attached Storage (NAS) 704a and 704b. FIG. 7 illustrates an example only, and a wide variety of other computer architectures and systems can be used in conjunction with the various embodiments of the present invention. For example, a blade server can be used to provide parallel processing. Processor blades can be connected through a back plane to provide parallel processing. Storage can also be connected to the back plane or as Network Attached Storage (NAS) through a separate network interface.

In some example embodiments, processors can maintain separate memory spaces and transmit data through network interfaces, back plane or other connectors for parallel processing by other processors. In other embodiments, some or all of the processors can use a shared virtual address memory space.

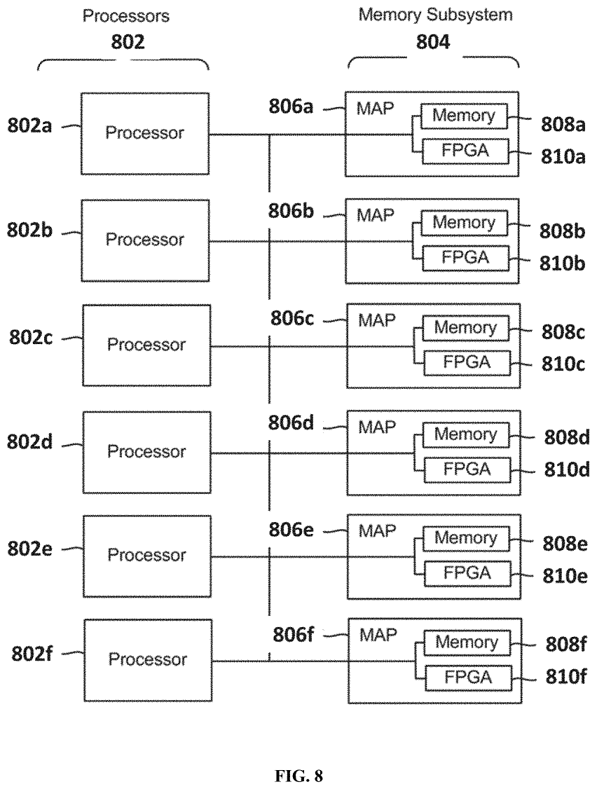

FIG. 8 is a block diagram of a multiprocessor computer system 800 using a shared virtual address memory space in accordance with an example embodiment. The system includes a plurality of processors 802a-f that can access a shared memory subsystem 804. The system incorporates a plurality of programmable hardware memory algorithm processors (MAPs) 806a-f in the memory subsystem 804. Each MAP 806a-f can comprise a memory 808a-f and one or more field programmable gate arrays (FPGAs) 810a-f. The MAP provides a configurable functional unit and particular algorithms, or portions of algorithms can be provided to the FPGAs 810a-f for processing in close coordination with a respective processor. For example, the MAPs can be used to evaluate algebraic expressions regarding the data model and to perform adaptive data restructuring in example embodiments. In this example, each MAP is globally accessible by all of the processors for these purposes. In one configuration, each MAP can use Direct Memory Access (DMA) to access an associated memory 808a-f, allowing it to execute tasks independently of, and asynchronously from, the respective microprocessor 802a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example embodiments, including systems using any combination of general processors, co-processors, FPGAs and other programmable logic devices, system on chips (SOCs), application specific integrated circuits (ASICs), and other processing and logic elements. In some embodiments, all or part of the computer system can be implemented in software or hardware. Any variety of data storage media can be used in connection with example embodiments, including random access memory, hard drives, flash memory, tape drives, disk arrays, Network Attached Storage (NAS) and other local or distributed data storage devices and systems.

In example embodiments, the computer system can be implemented using software modules executing on any of the above or other computer architectures and systems. In other embodiments, the functions of the system can be implemented partially or completely in firmware, programmable logic devices such as field programmable gate arrays (FPGAs), system on chips (SOCs), application specific integrated circuits (ASICs), or other processing and logic elements. For example, the Set Processor and Optimizer can be implemented with hardware acceleration through the use of a hardware accelerator card.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

### Example 1: Device Comprising TiN Reaction Chamber and Backplane Illumination

A device is functionalized to support the attachment and synthesis of polynucleotides. The device comprises an array of apertures, or reaction chambers. The individual apertures, or reaction chambers, are illuminated from the black pane of the device and are individually addressed through integrated CMOS. The dimensions of the reaction chambers are designed to eliminate crosstalk between features. Reaction chambers can be zeromode waveguides to confine the light in the reaction chamber.

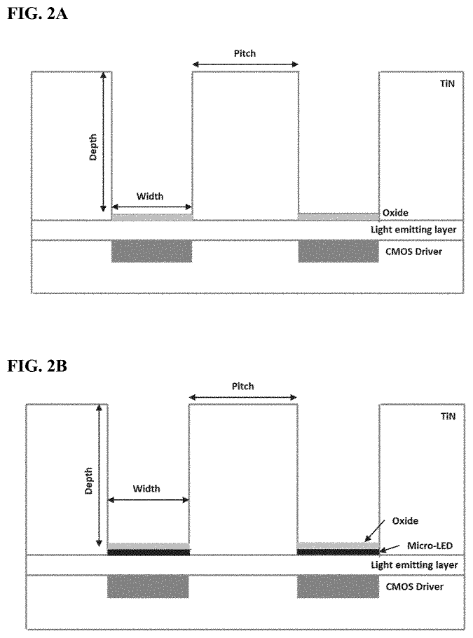

FIG. 2A illustrates a simplified cross-section diagram of an OLED light-directed polymer synthesis device with two reaction chambers. FIG. 2B illustrates a simplified cross-section diagram of a micro-LED light-directed polymer synthesis device with two reaction chambers. The reaction chamber is made of titanium nitride (TiN) and comprises two reaction chambers. The bottom of each reaction chamber comprises an oxide material, which is placed above a light-emitting layer. CMOS drivers are placed under, and in contact with, the light-emitting layer. The light-emitting layer of the reaction chambers is individually addressed to illuminate the individual reaction chamber.

### Example 2: Illumination of Individual Reaction Chambers

The illumination device of EXAMPLE 1 is used to synthesizes two oligonucleotide sequences. Logic input controls individual CMOS drivers, and the current generated results in photon emission from the light emitting layer, which removes the 5′-photolabile group and controls the sequence.

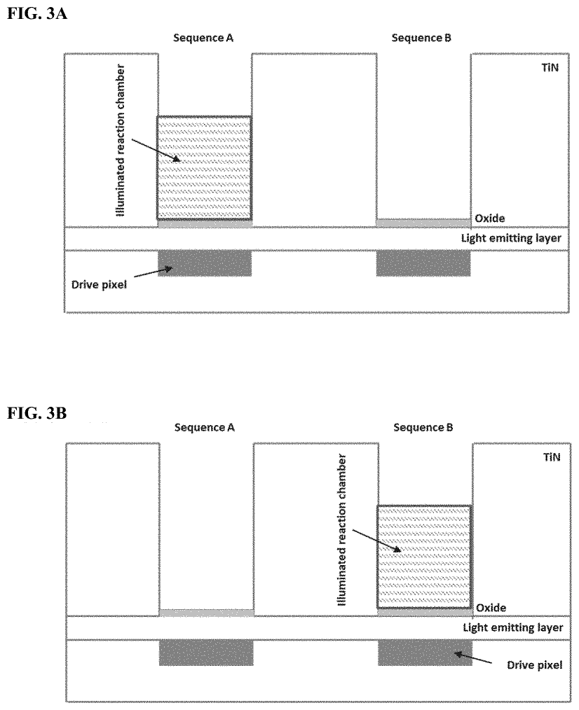

FIG. 3A illustrates photon emission of the CMOS driver on the left, which illuminates the reaction chamber on the left. The reaction chamber of the left is illuminated, and the deprotection step is used to synthesize Sequence A. FIG. 3B illustrates photon emission of the CMOS driver on the right, which illuminates the reaction chamber on the right. The reaction chamber on the right is illuminated, and the deprotection step is used to synthesize Sequence B.

### Example 3: Polymer Synthesis Device with a Light-Emitting Layer Comprising an OLED Stack

A polymer synthesis device using light-directed deprotection chemistry is built with an OLED stack. Organic light-emitting diodes (OLEDs) are monolithic, solid-state devices that consist of a series of thin films sandwiched between wo thin-film conductive electrodes. When electricity is applied to an OLED, under the influence of an electrical field, charge carriers (holes and electrons) migrate from the electrodes into the organic thin films until they recombine in the emissive zone forming excitons. Once formed, these excitons relax to a lower energy level by emitting light and/or unwanted heat.

A basic OLED cell structure consists of a stack of thin organic layers sandwiched between a conducting anode and a conducting cathode. The OLED structure comprises:
Substrate: foundation of the OLED (e.g., plastic, glass, or metal foil);
Anode: positively charged to inject holes (absence of electrons) into the organic layers that make up the OLED device; can be transparent;
Hole injection layer (HIL): deposited on top of the anode; HIL receives holes from the anode and injects them deeper into the device;
Light-emitting layer: consists of a color-defining emitter doped into a host; electrical energy is directly converted into light;
Blocking layer (BL): confines electrons (charge carriers) to the light-emitting layer;
Electron transport layer (ETL): supports the transport of electrons to light-emitting layer;
Cathode: negatively charged to inject electrons into the organic layers that make up the OLED device, can be transparent.

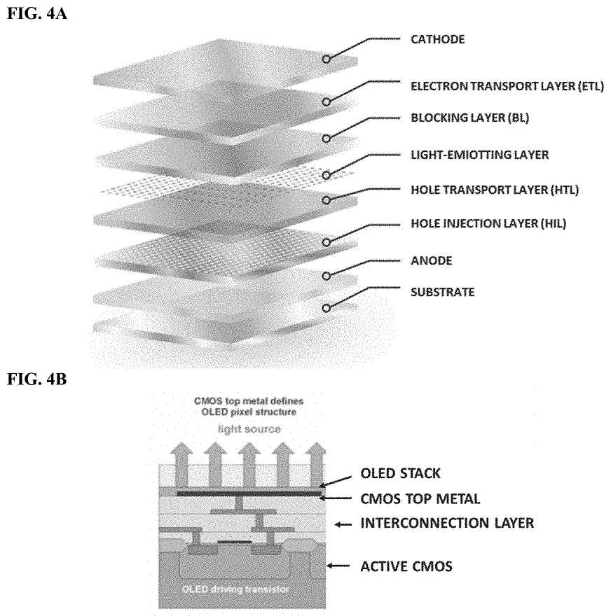

FIG. 4A illustrates the basic OLED cell structure comprising an anode, hole injection layer, light-emitting layer, blocking layer, electron transport layer, and cathode.

The OLED stack comprises 4 layers, from top to bottom: 1) an OLED stack; 2) CMOS top metal; 3) interconnection layer; and 4) active COMS (OLED-driving transistor). Each layer is in contact with the subsequent layer, and the reaction chambers are fabricated above the light-emitting OLED stack. The reaction chambers are fabricated by depositing a thin layer of SiO₂, followed by TiN, and then etching through the TiN layer and stopping on the SiO₂ layer. The SiO₂ surface can be selectively functionalized for subsequent DNA synthesis chemistry. TiN can be specifically passivated to prevent fouling. The desired wavelength is obtained by choosing a specific organic material for the top layer, and light is illuminated into the reaction chamber (arrows). The CMOS top metal defines the OLED pixel structure.

FIG. 4B illustrates the OLED example stack of the disclosure comprising an OLED stack, CMOS top metal layer, an interconnection layer, and an active CMOS.

### Example 4: Polymer Synthesis Device with a Light-Emitting Layer Comprising a Micro-LED Stack

A polymer synthesis device using light-directed deprotection chemistry is built with a micro-LED structure. A micro-LED panel includes a single crystalline Si substrate (1302) and a plurality of driver circuits (1304) fabricated at least partially in the substrate (1302). Each of the driver circuits (1304) includes a MOS-based integrated circuit. The driver circuit (1304) can have more than one MOS structure. The MOS structure (1306) includes a first source/drain region (1306-1), a second source/drain region (1306-2), and a channel region (1306-3) formed between the first and second source/drain regions. The MOS structure (1306) further includes a gate (1306-4) and a gate dielectric layer (1306-5) formed between the gate (1306-4) and the channel region (1306-3). First and second source/drain contacts (1306-6 and 1306-7) are formed to be electrically coupled to the first and second source/drain regions (1306-1 and 1306-2, respectively) for electrically coupling the first and second source/drain regions (1306-1 and 1306-2) to other portions of the LED panel.

The micro-LED stack has a 12×9 mm display with 2.5 μm pitch, and 5000 rows×4000 columns. An active CMOS backplane is bonded to an InGaN/AlGaN wafer to achieve 365 nm illumination wavelength. The reaction chambers are fabricated above the light emitting layer. The reaction chambers are fabricated by depositing TiN, etching through the TiN layer to form a well or flow cell, and depositing an oxide at the bottom of the well or flow cell. The reaction chambers can also be fabricated by depositing TiN, etching through the TiN layer to form a well or flow cell and directly functionalizing the well or flow cell with a micro-LED. The SiO₂ surface can be selectively functionalized for subsequence DNA synthesis chemistry. TiN can be specifically passivated to prevent fouling.

The reaction chambers are fabricated above the light-emitting OLED stack. The reaction chambers are fabricated by depositing a thin layer of SiO₂, followed by TiN, and then etching through the TiN layer and stopping on the SiO₂ layer. The SiO₂ surface can be selectively functionalized for subsequence DNA synthesis chemistry. TiN can be specifically passivated to prevent fouling. The desired wavelength is obtained by choosing a specific organic material for the top layer. Light is illuminated into the reaction chamber (arrows). The CMOS top metal defines the OLED pixel structure.

### Example 5: Device Comprising GaN Reaction Chamber

A polymer synthesizer is developed using GaN semiconductors. External quantum efficiency (EQE) is a quantity defined for a photosensitive device as the percentage of photons hitting a photoreactive surface that will produce an electron-hold pair. EQE is an accurate measurement of a device's electrical sensitivity to light. The total brightness of the device is >30× OLEDs, and the external quantum efficiency (EQE) is greater than 70%. The device allows for a pixel pitch of less than 100 nm. Bonding the GaN wafer to a CMOS backplane allows the development of wafers up to 300 mm in size. CMOS chips with up to 4 megapixels and 2.5 μm are used.

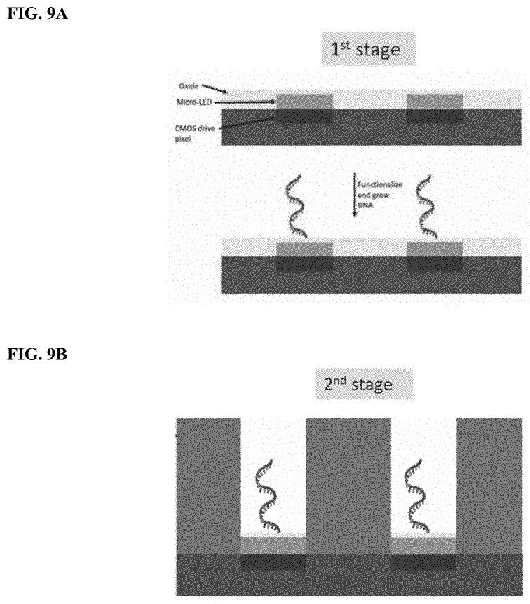

Specifications of the GaN microLED chip are shown in FIG. 9A and FIG. 9B. A SiO₂ layer is used to cover the GaN microLED, see FIG. 9A. The surface of the chip is functionalized with CVD, followed by a very thin DNA film (nm). A flow cell is attached to the surface to allow the fluidics to work. The LED wavelength is 405 nm. The second stage of the microLED chip isolates pixels by forming walls that are 400 nm thick, see FIG. 9B.

### Example 6: GaN on Si MicroLED Polymer Synthesis Device

A GaN on Si microLED polymer synthesis device was prepared. A SiO₂ surface was functionalized with glycidoxypropyl trimethoxysilane (GOPS) for use in DNA synthesis. TABLE 1 shows pixel and emission area measurements of the GaN on Si microLED polymer synthesis device.

TABLE 1

| | | Array Pixel | | | |
|---|---|---|---|---|---|
| | Number of | LR | Array | Total LR | Total BL |
| | Arrays on | Diameter | pixel | Emission | Emission |
| Array | Device | (μm) | count | Area (μm²) | Area (μm²) |
| A9 | 25 | 1 | 4225 | 3318 | 53093 |

The GaN on Si chip was used to grow an epitaxial layer to achieve 405 nm emission. 450 nm of oxide was deposited on GaN to give roughened and unroughened surfaces for testing. Both the unroughened and roughened devices suffered from poor uniformity. The poor uniformity of the devices resulted from the pixel arrays being connected in parallel. The dyes were voltage driven, and any minor variation in voltage caused pixel bright spots.

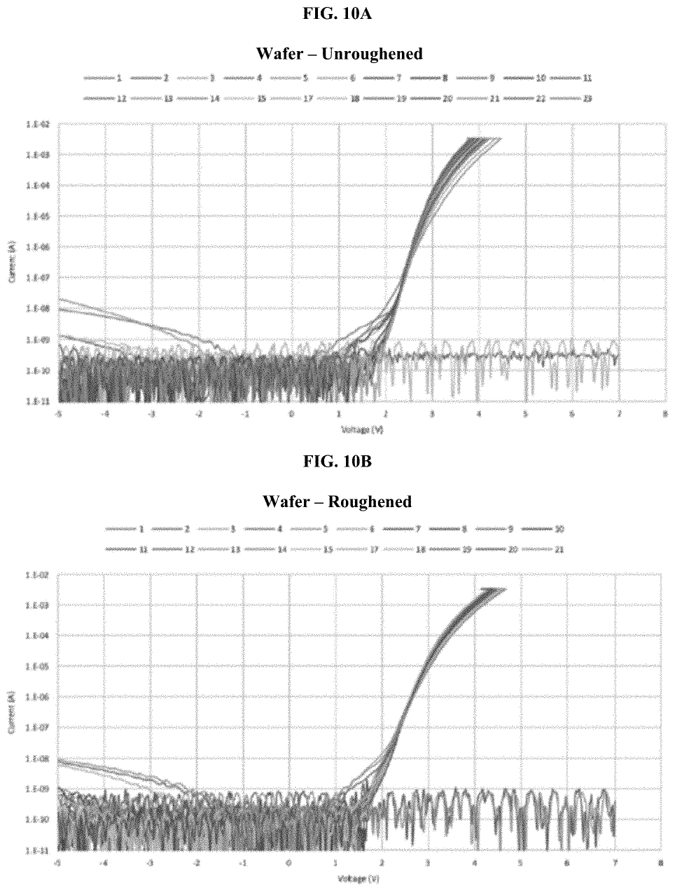

FIG. 10A shows DC I-V plots of a wafer with an unroughened surface. FIG. 10B shows DC I-V plots of a wafer with a roughened surface. Each wafer had 25 chips, and only a few of the chips had bonding or packaging issues. The rest of the chips showed consistent behavior, as demonstrated by the I-V plots of FIGS. 10A and 10B. More light (i.e., external quantum efficiency; EQE) was observed from the roughened devices compared to the unroughened devices.

Peak EQE measurements for 405 nm epi-fluorescence was about 10% for wafers with unroughened surfaces and 17% for wafers with roughened surfaces. TABLE 2 shows the results of EQE measurements for two wafer samples. The data show that the devices with roughened surfaces (i.e., TABLE 2, wafer 1) had higher EQE than devices with unroughened surfaces (i.e., TABLE 2, wafer 2). On average, the devices with roughened surfaces had peak EQEs (%) that were about 1.8-fold higher than the peak EQEs of devices with unroughened surfaces.

TABLE 2

| | | Radiant Efficiency | | | Luminous Efficiency | | | EQE | | |
|---|---|---|---|---|---|---|---|---|---|---|
| | | Peak | J | If | Peak | J | IF | | J | IF |
| Wafer | Device | (%) | (A/cm²) | (mA) | (lm/W) | (A/cm²) | (mA) | Peak | (A/cm²) | (mA) |
| 1 | 2 | 9.316 | 31.85 | 16.91 | 0.640 | 31.85 | 16.91 | 0.135 | 124.1 | 65.90 |
| | 3 | 9.624 | 61.95 | 32.89 | 0.660 | 61.95 | 32.89 | 0.138 | 124.1 | 65.90 |
| | 4 | 9.124 | 61.95 | 32.89 | 0.630 | 31.85 | 16.91 | 0.134 | 124.1 | 65.90 |
| | 5 | 9.437 | 61.95 | 32.89 | 0.660 | 61.95 | 32.89 | 0.137 | 124.1 | 65.89 |
| | 6 | 9.743 | 61.95 | 32.89 | 0.670 | 31.85 | 16.91 | 0.141 | 124.1 | 65.90 |
| 2 | 2 | 17.51 | 31.85 | 16.91 | 1.200 | 31.85 | 16.91 | 0.270 | 124.1 | 65.89 |
| | 3 | 16.40 | 31.85 | 16.91 | 1.140 | 31.85 | 16.91 | 0.263 | 61.95 | 32.89 |
| | 4 | 17.00 | 31.85 | 16.91 | 1.170 | 31.85 | 16.91 | 0.259 | 61.95 | 32.89 |
| | 5 | 16.92 | 31.85 | 16.91 | 1.140 | 31.85 | 16.91 | 0.259 | 124.1 | 65.89 |
| | 6 | 16.93 | 31.85 | 16.91 | 1.170 | 31.85 | 16.91 | 0.257 | 61.95 | 32.89 |

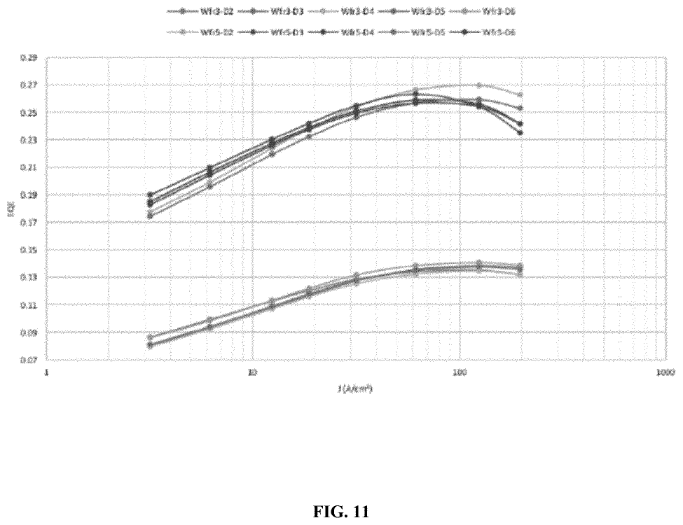

FIG. 11 shows peak external quantum efficiency measurements for 10 wafer samples. EQE is the resulting photon flux divided by the LED electron flux. The EQE was measured on an integrating sphere.

### Example 7: Surface Chemistry and Fluidics Required for Synthesis

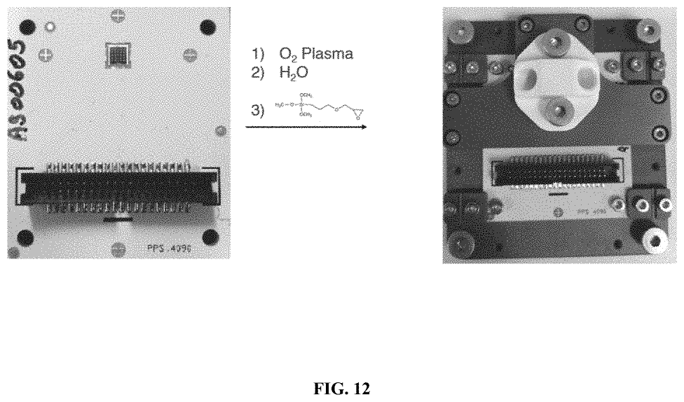

FIG. 12 (left) shows an image of the packaged chip. FIG. 12 (right) shows an image of a fluidics system required for DNA synthesis. Surface coating was performed using 02 plasma, water, and trimethoxy(3-(oxiran-2-ylmethoxy)propyl)silane. Only the middle 9 arrays were wired due to flow cell trade off.

### Example 8: Synthesis of 5′-Photolabile dT Amidites

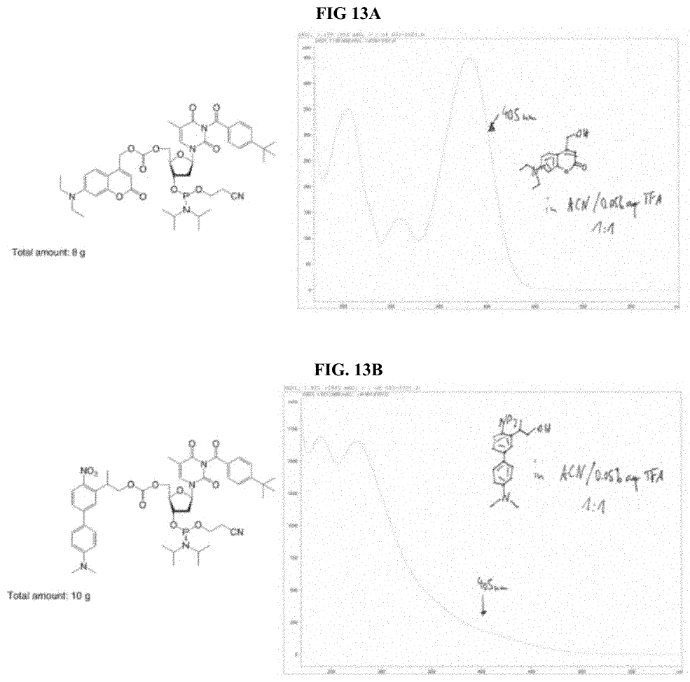

FIGS. 13A and 13B show UV spectra of 5′-photolabile dT amidites cleavable at 405 nm. FIG. 13A shows the UV spectrum of ((2R,3S,5R)-5-(3-(4-(tert-butyl)benzoyl)-5-methyl-2,4-dioxo-3,4-dihydropyrimidin-1(2H)-yl)-3-(((2-cyanoethoxy)(diisopropylamino)phosphaneyl) oxy)tetrahydrofuran-2-yl)methyl ((2-(diethylamino)-7-oxo-7,8-dihydro-1λ³-chromen-5-yl)methyl) carbonate. FIG. 13B shows the UV spectrum of ((2R,3S,5R)-5-(3-(4-(tert-butyl)benzoyl)-5-methyl-2,4-dioxo-3,4-dihydropyrimidin-1(2H)-yl)-3-(((2-cyanoethoxy)(diisopropylamino)phosphaneyl)oxy)tetrahydrofuran-2-yl)methyl (2-(4′-(dimethylamino)-4-nitro-[1,1′-biphenyl]-3-yl)propyl) carbonate.

### Example 9: Proof of Concept Experiments

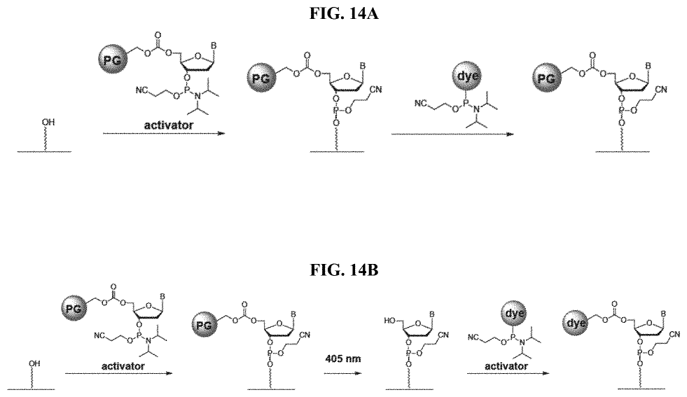

FIG. 14A shows the chemical reactions of the control experiment. A surface comprising hydroxyl groups is reacted with a phosphoramidite activating group comprising a protecting group (PG). The surface-linked protected nucleotide is further reacted with a phosphoramidite group comprising a dye. In the control experiment, the surface-linked, protected nucleotide group remains unreacted.

FIG. 14B shows the chemical reactions of the proof of concept experiment. A surface comprising hydroxyl groups is reacted with a phosphoramidite activating group comprising a protecting group (PG). The surface-linked protected nucleotide is photo-deprotected by illuminating the surface-linked protected nucleotide moiety at 405 nm. The deprotected nucleotide is further reacted with a dye-linked phosphoramidite activator. In the proof of concept experiment, the surface-linked nucleotide is labeled with the dye.

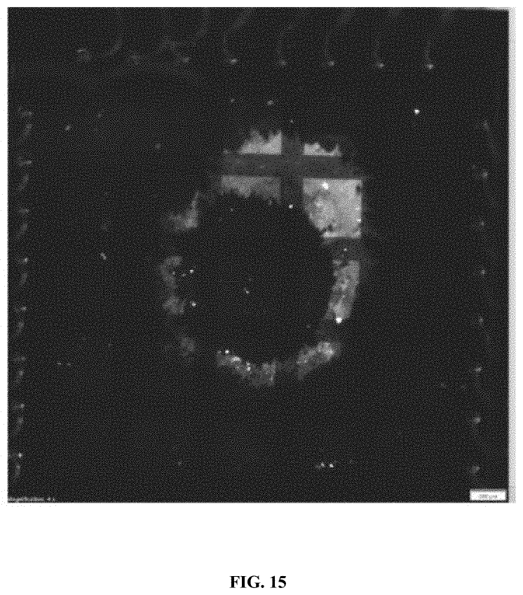

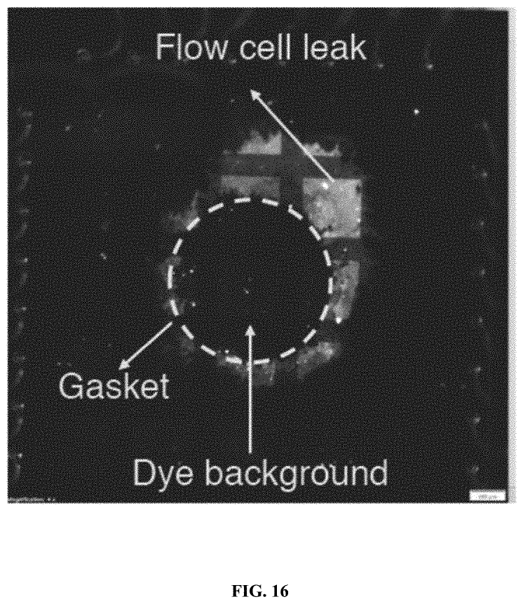

FIG. 15 shows an image of the control reaction of FIG. 14A performed using on-chip 1 μm microLED DNA synthesis. The dye was Cy3. FIG. 16 shows that the control reaction of FIG. 14A and FIG. 15 had flow cell leakage, and the dye was visualized as the background.

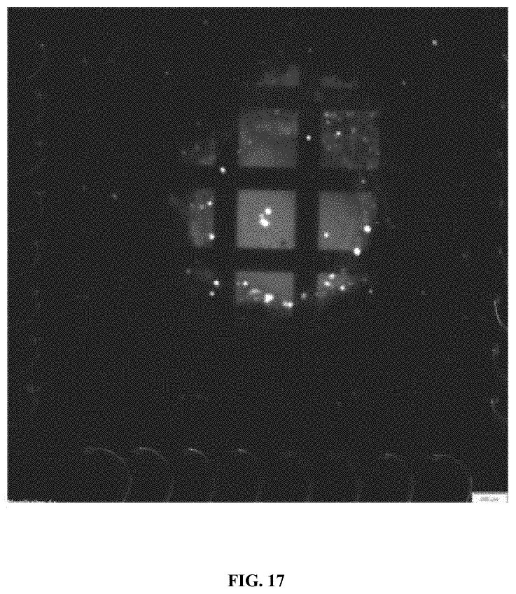

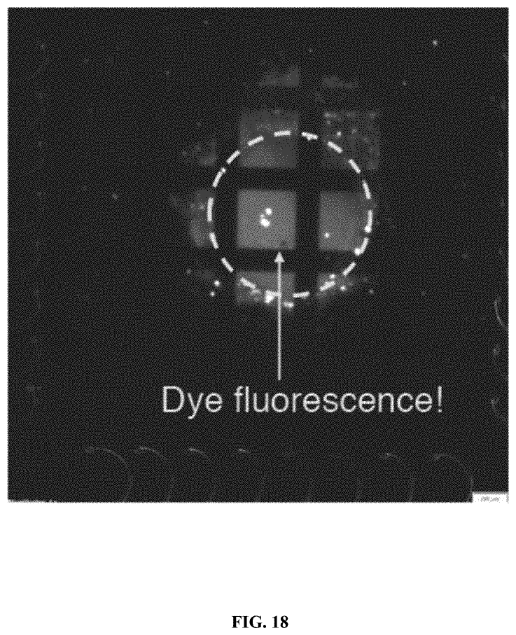

FIG. 17 shows an image of the proof of concept reaction of FIG. 14B performed using on-chip 1 μm microLED DNA synthesis. FIG. 18 shows that the experiment of FIG. 14B and FIG. 17 resulted in dye fluorescence after 1 min exposure 4V.

### Example 10: Patterning Chips with Walls or Building Relay Lenses for Crosstalk Testing

A device is prepared where DNA synthesis is not conducted directly on a chip. A light source is decoupled from the chip, and DNA synthesis is carried out on a disposable substrate (e.g., glass substrate). A microLED COMS chip is patterned with walls that are about 400 nm thick and/or a relay lens is built for crosstalk testing.

1. A device for polymer synthesis, comprising:
a solid support, wherein the solid support comprises a plurality of wells, wherein each of the wells comprises:
a) a synthesis surface located in a bottom region of each of the wells;
b) a light-emitting layer in addressable communication with the synthesis surface and situated below the synthesis surface; and
c) a CMOS driver located in addressable communication with the light-emitting layer.

2. The device of claim 1, wherein the light-emitting layer is a light-emitting diode (LED).

3. The device of claim 2, wherein the LED is an organic LED (OLED) or a micro-LED.

4. (canceled)

5. The device of claim 1, wherein the light-emitting layer emits ultraviolet (UV) light visible light, or infrared (IR) light.

6. The device of claim 5, wherein the UV light has a wavelength of about 350, 365, or 400 nm.

7-9. (canceled)

10. The device of claim 5, wherein the visible light has a wavelength of about 405 or 450 nm.

11. The device of claim 5, wherein the visible light has a wavelength of about 450 nm.

12. (canceled)

13. The device of claim 5, wherein the IR light has a wavelength of about 800 nm.

14. The device of claim 1, wherein the solid support comprises addressable loci at a density of at least 10×10⁶ addressable loci per cm².

15. (canceled)

16. The device of claim 1, wherein the solid support comprises addressable loci, and each addressable locus comprises a diameter up to about 1000 nm.

17. (canceled)

18. The device of claim 1, wherein each of the wells comprises a depth of 100 nm to 1000 nm.

19-22. (canceled)

23. A method for synthesizing a polymer, comprising:
a) providing a solid support comprising a surface;
b) depositing at least one nucleoside on the surface, wherein the at least one nucleoside couples to a polynucleotide attached to the surface, wherein the coupling comprises a light-directed deprotection step by a light-emitting layer, and wherein the light-emitting layer is located beneath the surface; and
c) repeating step b) to synthesize a plurality of polynucleotides on the surface, wherein polynucleotides having different sequences on the surface are present at a density of at least 100×106 polynucleotides per cm².

24. The method of claim 23, wherein the light-emitting layer is a light-emitting diode (LED).

25. The method of claim 24, wherein the LED is an organic LED (OLED) or a micro-LED.

26. (canceled)

27. The method of claim 23, wherein the light-emitting layer emits ultraviolet (UV) light, visible light, or infrared (IR) light.

28. The method of claim 27, wherein the light has a wavelength of about 350, 365, 400, 405, 450, or 800 nm.

29-38. (canceled)

39. The method of claim 23, wherein the deprotection step deprotects a 5′-hydroxyl group.

40. The method of claim 39, wherein the 5′-hydroxyl group is protected by a protecting group of the formula: 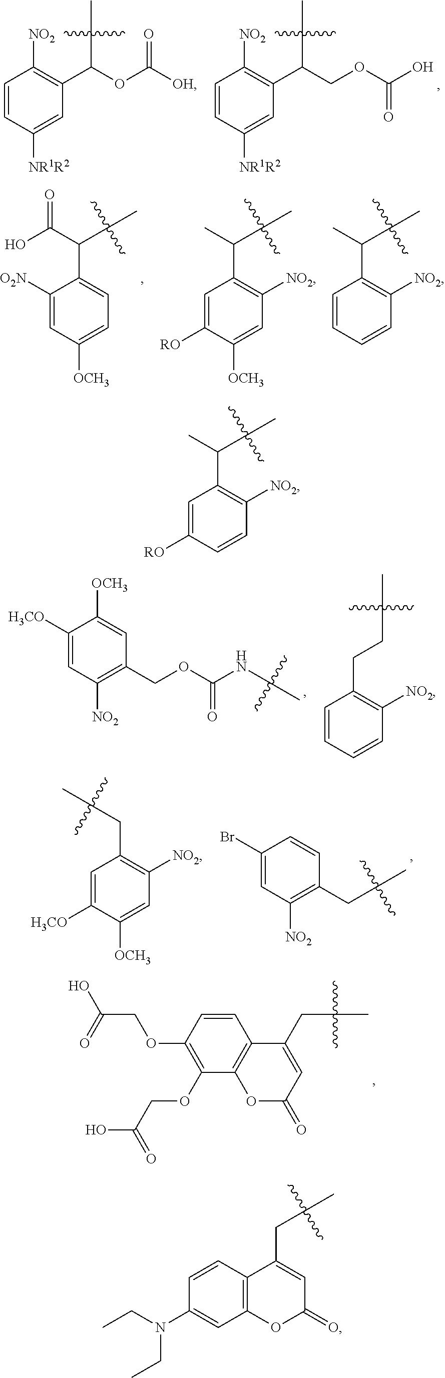 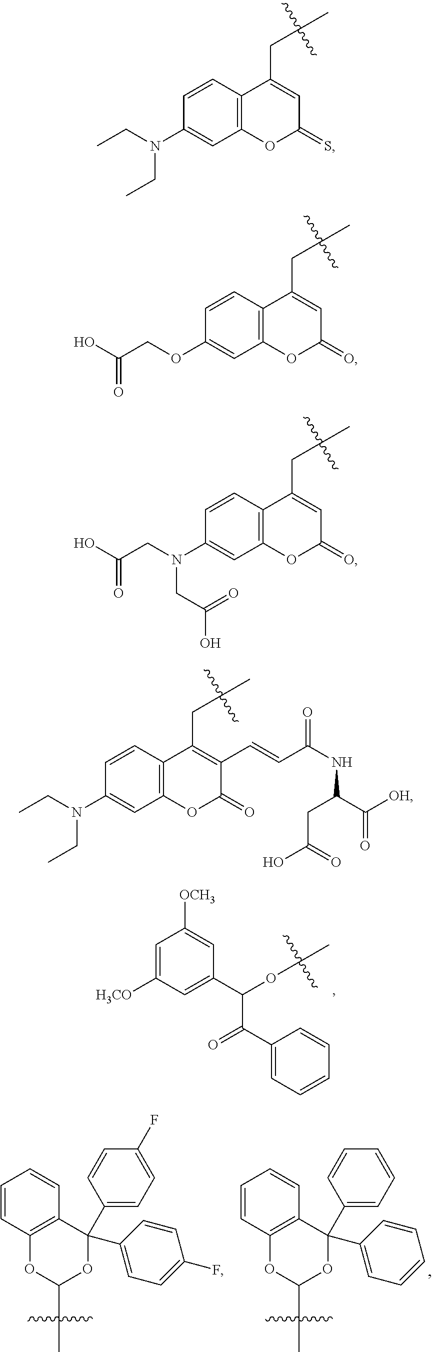 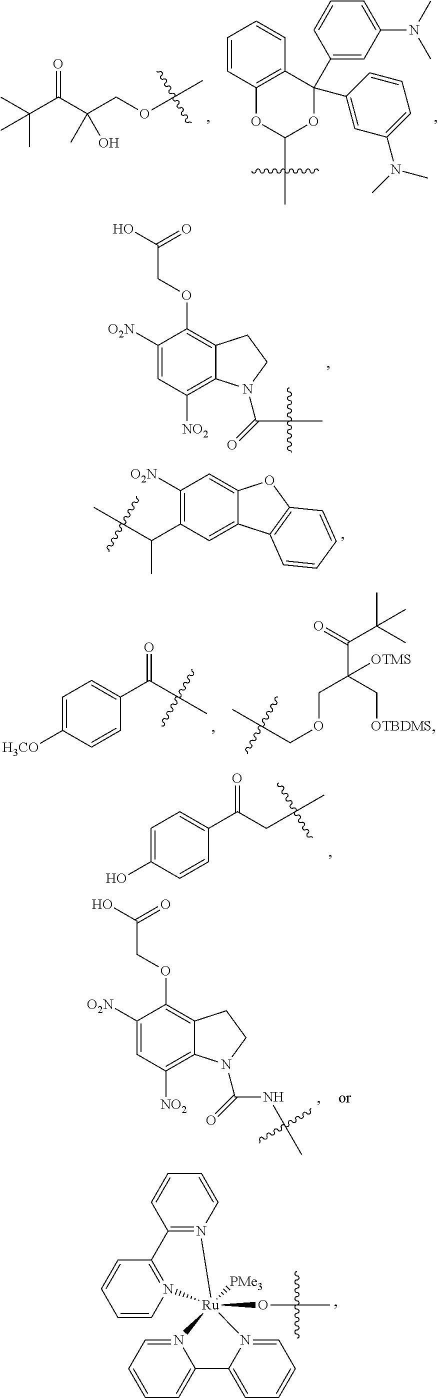
wherein each R, R¹, and R² is independently is selected from a group consisting of: —C(O)R³, —C(O)OR³, —C(O)NR³R⁴, —SOR³, —SO₂R⁴, alkyl, alkenyl, alkynyl, aryl, heteroaryl, or heterocyclyl, each of which is independently substituted or unsubstituted, or hydrogen, or R¹ and R² together with the nitrogen atom to which R¹ and R² are bound form a ring, wherein the ring is substituted or unsubstituted; wherein each R³ and R⁴ is independently —C(O)R⁵, —C(O)OR⁵, —C(O)NR⁵R⁶, —OR⁵, —SR⁵, —NR⁵R⁶, —NR⁵C(O)R⁶, —OC(O)R⁵, alkyl, alkenyl, alkynyl, aryl, heteroaryl, or heterocyclyl, each of which is independently substituted or unsubstituted, or hydrogen or halogen; wherein each R⁵ and R⁶ is independently alkyl, alkenyl, alkynyl, aryl, heteroaryl, or heterocyclyl, each of which is independently substituted or unsubstituted, or hydrogen.

41. The method of claim 39, wherein the 5′-hydroxyl group is protected by a nitrophenylpropyloxycarbonyl (NPPOC) protecting group.

42. The method of claim 39, wherein the 5′-hydroxyl group is protected by a 2,(3,4-methylenediooxy-6-nitrophenyl)propoxycarbonyl (MNPPOC) group.
